(19) United States
(12) Patent Application Publication
(10) Pub. No.: US 2007/0098801 A1
(43) Pub. Date: May 3, 2007

(54) Particles shaped as platelets

(75) Inventors: Geert Verreck, Zoersel (BE); Albertina Maria Eduarda Arien, Lille (BE); Jozef Peeters, Beerse (BE); Marcus Eli Brewster, Beerse (BE); David Lane Tomasko, Columbus, OH (US); Hungbo Li, Laval (CA)

Correspondence Address: PHILIP S. JOHNSON;JOHNSON & JOHNSON, ONE JOHNSON & JOHNSON PLAZA, NEW BRUNSWICK, NJ, 08933-7003, US

(73) Assignee: JANSSEN PHARMACEUTICA N.V., Beerse (BE)

(21) Appl. No.: 10/571,663

(22) PCT Filed: Sep. 9, 2004
(86) PCT No.: PCT/EP04/52104

§ 371 Date: Oct. 27, 2006

Related U.S. Application Data
(60) Provisional application No. 60/501,639, filed on Sep. 10, 2003.

(51) Int. Cl. A61K 31/497; A61K 31/496; A61K 9/14

(52) U.S. Cl. 424/489; 514/253.07

(57) ABSTRACT

The present invention relates to polymer particles shaped as platelets and to a process of manufacturing such particles. The particles according to the invention exhibit a faster rate of dissolution in aqueous media than art-known particles.

The present invention relates to polymer particles shaped as platelets and to a process of manufacturing such particles. The particles according to the invention exhibit a faster rate of dissolution in aqueous media than art-known particles. The invention further concerns polymer particles comprising an active ingredient, pharmaceutical dosage forms and a process of manufacturing such dosage forms.

### I. INTRODUCTION

The utilization of supercritical fluids (SCF) for the processing of pharmaceuticals has gained considerable interest in the last decade. Supercritical fluids can be used, (a) to extract substances from natural sources, (b) as solvents or anti-solvents for particle engineering, encapsulating drugs into polymeric carriers, resolving racemic mixtures of active compounds or fractionating mixtures of polymers or proteins, (c) as reaction medium for chemical reactions, and (d) to sterilize bacterial organisms (1-3). The most important advantages of materials processed using supercritical fluid techniques in pharmaceutical areas include the high quality of the products in terms of purity, their unique morphology, and the wide range of substances that can be processed. Also, supercritical fluids based on carbon dioxide are environmentally friendly, whereas conventional pharmaceutical processes are often associated with both the emissions of organic solvents and with difficulties of removing residual solvent. Furthermore, the mild operating conditions associated with supercritical carbon dioxide can be especially favourable to bio-molecules, such as proteins involved in pharmaceutical applications. As indicated, the most popular supercritical fluid in pharmaceutical applications is carbon dioxide. It is non-toxic, non-flammable, tasteless, inert and inexpensive, which makes carbon dioxide a perfect substitute for organic solvents.

One of the original applications of supercritical fluids, i.e. particle engineering and particle formation processes, are becoming more and more popular in the pharmaceutical industry (4). One major reason for this increased interest, can be found in the ability of these techniques to encapsulate drugs into polymeric carriers, i.e. the formation of a solid dispersion through the use of supercritical fluids.

More and more drug candidates tend to have higher molecular weights, high lipophilicities, and low aqueous solubilities, usually resulting in poorer oral bioavailability (5-7). In assessing methods to provide for an orally bioavailable formulation of poorly water soluble drug candidates, a useful starting point is the Noyes-Whitney equation which describes the dissolution rate of a solid:

$$\frac{ⅆ C}{ⅆ t} = \frac{D \times A \times ( {C}_{s} - {C}_{t} )}{h \times V}$$

where the dissolution rate (dC/dt) is determined by D, the diffusion coefficient, h, the diffusion layer thickness at the solid-liquid interface, A, the surface area of drug exposed to the dissolution medium, V, the volume of the dissolution medium, Cₛ, the saturation solubility of the drug in the dissolution medium and Cₜ, the drug concentration at time, t. In other words, dissolution rate can be increased by (a) increasing the surface area of the drug (via micro- or nanosizing), (b) by decreasing the diffusional layer thickness (through improving wettability by e.g. addition of surfactants) and, (c) by altering the solubility of the drug (through formation of supersaturated drug solution via solid dispersion, complexation approaches or by manipulation of the solid form to give more soluble salts, polymorphs or amorphous material).

The solid dispersion approach is generally accepted as a possible method to increase aqueous solubility and eventually oral bioavailability (8-10). Two common methods exist to prepare solid dispersions: (a) the solvent method, and (b) the hot melt method (9, 10). With the solvent method, the drug and carrier are dissolved in a common organic solvent, followed by removal of the solvent by evaporation. This can be done, for instance, by spray-drying, whereby the solution is pumped into a chamber through a spraying nozzle, and is then distributed into a fine mist of small droplets. The solvent is rapidly evaporated from these small droplets and particles are collected in a cyclotron. The hot melt method consists of melting the carrier and drug whereby the solid dispersion is formed upon cooling of the melt. In some cases it is sufficient to melt only the carrier, and dissolve/disperse the crystalline drug in the molten carrier. Different approaches exist to perform the hot melt method, but during the last decade, hot melt extrusion, which originates from the polymer processing industry, was developed for some pharmaceutical applications and gained since then increasing popularity (11, 12). It is a well-known advantage of the hot melt extrusion process over any solvent method that the formation of such a solid dispersion is solvent free (13). Indeed with solvent processes, a number of concerns related to environmental pollution, explosion-proofing and residual solvent may arise. On the other hand, one major drawback of hot melt extrusion is the long residence time at increased temperature. This would exclude the application of hot stage extrusion for thermo-labile compounds or proteins that need to be dispersed into a polymeric carrier.

These facts point to a possible synergy between supercritical fluid technology and hot melt extrusion. The properties of a supercritical fluid, i.e. (a) a solvent for the thermo-labile active and (b) a plasticizer for the polymeric carrier, would expand the applicability of hot melt extrusion for use with thermo-labile compounds. Therefore, the aim of the current research project is to evaluate whether pressurized carbon dioxide can be injected into the hot melt extruder (importance of the design of the extruder set up) and as such will act as a plasticizer and foaming agent for the polymeric carrier.

1. Physical chemistry of a supercritical fluid.

The thermodynamic condition of a single substance is defined by the variables of pressure (p), temperature (T) and volume (V) which results in a three-dimensional phase diagram. A simplified presentation of this phase diagram can be obtained by projecting the p and T axis (2, 14). This graph depicts the borders between the different phases: solid, liquid and gas, where all three phases coexist in the triple point. If one moves upwards along the gas-liquid coexistence curve, both temperature and pressure increase. The liquid becomes less dense as the temperature increases and the gas becomes more dense as the pressure increases. Eventually, the densities of the two phases become identical, i.e. the distinction between the gas and the liquid disappears with the curve terminating at the critical point The coordinates of the critical point are referred to as the critical temperature and critical pressure, and which have discrete values for particular substances, as shown in Table 1.

In table 1 and throughout the specification, the unit ‘bar’ corresponds to the SI unit ‘Pa’ according to the following equation: 1 bar=100 kPa.

TABLE 1
Critical parameters for a few substances

| Substance | Critical temperature (K) | Critical pressure (bar) |
|---|---|---|
| carbon dioxide | 304.1 | 73.8 |
| ethane | 305.4 | 48.8 |
| ethene | 282.4 | 50.4 |
| propane | 369.8 | 42.5 |
| propene | 364.9 | 46.0 |
| trifluoromethane | 299.3 | 48.6 |
| ammonia | 405.5 | 113.5 |
| water | 647.3 | 221.2 |
| cyclohexane | 553.5 | 40.7 |
| n-pentane | 469.7 | 33.7 |
| toluene | 591.8 | 41.0 |
| xylene | 616.1 | 35.2 |
| xenon | 289.7 | 58.4 |

As the critical point of a substance is approached, its isothermal compressibility approaches infinity, and thus, its molar volume, or density, changes dramatically. A supercritical fluid can provide the solvent capacity of classical solvents, while providing higher diffusional capacity through its proximity to the gas state. The physicochemical parameters shown in Table 2 demonstrate the specific properties of the supercritical fluids which are often viewed as “dense gases”.

TABLE 2
Density, viscosity and diffusion coefficient ranges
of liquids, gases and supercritical fluids.

| | Liquid | SCF | Gas |
|---|---|---|---|
| Density (kg/m³) | 1000 | 100-800 | 1 |
| Viscosity (Pa · s) | 10⁻³ | 10⁻⁵-10⁻⁴ | 10⁻⁵ |
| Diff. Coef.: m²/s | 10⁻⁹ | 10⁻⁸ | 10⁻⁵ |

The density of a supercritical fluid will increase with pressure at constant temperature. On the other hand, the density will decrease when temperature is increased at constant pressure. Close to the critical point, density changes are considerable and thus the solubility of a substance can be tailored by fine tuning pressure and temperature.

The diffusivity of a supercritical fluid will increase at increasing temperature and/or decreasing pressure. Again the largest changes in diffusivity occur in the vicinity of the critical point.

Finally, the viscosity of a supercritical fluid will behave in a manner similar to its density. In other words, viscosity will increase at increasing pressure and/or decreasing temperature.

The most important advantages of supercritical carbon dioxide include: (a) selectivity, since small changes in pressure and temperature may result in large changes of the properties, (b) no residual solvent, since the supercritical fluid returns to the gaseous phase upon releasing the pressure and temperature, (c) the critical temperature of carbon dioxide is low (31° C.), which allows the processing of thermo-labile materials, (d) it is non-toxic, inert and non-flammable, and (e) it is inexpensive.

The most important disadvantages of supercritical carbon dioxide include: (a) the need for a relatively high pressure (73.8 bar), (b) recycling of carbon dioxide is expensive and requires complex equipment, and (c) the application in pharmaceutical industry is relatively new and thus the cost of equipment and investment is high.

2. Pharmaceutical applications of supercritical fluids.

A number of applications of supercritical fluids in pharmaceutical industry exist today. At present, most industrial pharmaceutical applications have focused on extraction/fractionation of natural products, e.g. residual solvents or other impurities, like pesticides, are extracted from active compounds at large scale (as in the case of ginseng preparation) (3). Another interesting application is the fractionation of proteins (e.g. insulin) using vapour-phase carbon dioxide (15). Supercritical fluid chromatography and preparative scale SCF chromatography have been developed for a number of applications, e.g. the fractionation of lipids like polyunsaturated fatty acids (16).

Besides extraction/fractionation, particle formation/engineering is currently one of the most popular applications of supercritical fluids in the area of pharmaceuticals. This technique can be further subdivided in three main research areas: (a) the preparation of powders of active substances to improve or modify their therapeutic action or to enhance their solubility (micronisation), (b) the production of polymers as a matrix for drug impregnation, and (c) the preparation of drug based polymeric carrier systems as drug delivery systems with improved bio-availability (i.e. solid dispersions) or sustained release characteristics. A number of processes for particle formation are cited in literature, but the most popular ones include the following:

RESS

In rapid expansion of supercritical solutions (RESS), a substance is dissolved in the SCF and sprayed into a low-pressure vessel. Solubility of the product in SCF should not be too low (≧10⁻³ kg/kg), limiting the application to non-polar products. Major advantages include the ability to produce very fine particles with controllable particle size in the absence of organic solvents. Major disadvantages include the high gas/substance ratio needed owing to the low solubility of the substance and the requirement of a large-volume pressurized equipment. A number of examples are cited in literature, such as the encapsulation of naproxen in L- polylactic acid (L-PLA) described by Tomasko et al. (17).

SAS/GAS

The supercritical anti-solvent (SAS, GAS) process can be applied to molecules which are soluble in a very wide range of organic solvents. The solid of interest is dissolved in a solvent after which a supercritical fluid, having low solvent power with respect to the solid but miscible with the solvent, is added to precipitate the solid. The process can be done batch wise (in a pressurized vessel) or in a continuous mode (spraying the solution into the supercritical fluid). A major advantage of the technique is that very fine particles are obtained with controllable particle size. The disadvantage is the use of solvents. Again, numerous examples are described in literature. For instance the precipitation of insulin by the GAS process as described by Debenedetti et al. (18).

SEDS

Solution enhanced dispersion by supercritical fluids (SEDS) is derived from the GAS process. With SEDS, the solution and SCF are combined in a specially designed nozzle and sprayed into a pressure vessel (19).

PGSS

In particle generation from supercritical solutions or suspensions (PGSS), the compressible medium is solubilized in the substance to be micronised. The gas-containing solution is then rapidly expanded in an expansion unit and the gas is evaporated. Advantages of the process include the formation of fine particles with a narrow particle size distribution and extremely low gas consumption. Nifedipine, for example, can be micronised by the PGSS process (20).

Impregnation

High diffusivity and tunable density/solvent power of a supercritical fluid form the basis of the impregnation process. Polymeric non-porous matrixes, tend to swell when exposed to supercritical fluids and thus the penetration of the solute through the solid is enhanced. Many impregnation applications have been reported including pharmaceutical patches, sponges, and catheters (21).

It is clear that a number of processes exist to disperse a drug in a polymeric carrier by supercritical fluids. Some of these processes represent only small variations on a theme and they all have their particular advantages and disadvantages. This area of research is relatively novel and new methods to disperse drugs in polymers by supercritical fluid treatment continues to be highly sought for by pharmaceutical research groups. From this point of view it was decided to explore and investigate the combined possibilities of supercritical fluids and hot melt extrusion.

3. Hot melt extrusion and pharmaceutical applications.

Hot melt extrusion is a common processing modality in the polymer industry. About 35 years ago, the process was adapted for pharmaceutical applications by Speiser (22). However, only in the last decade has the process gained significant interest such that it is now generally accepted in pharmaceutical industry as a valuable technique to prepare solid dispersions.

The process can be divided into four aspects: (a) feeding of the powders into the extruder, (b) conveying the polymer or drug/polymer mass and entry into the die, (c) flow through the die, and (d) exit from the die and down-stream processing (12, 23, 24). Important considerations include: powder flowability, shear force, residence time, pressure, cooling and shaping. Generally, the extruder consists of at least one rotating screw inside the barrel. An end-plate die, connected to the end of the barrel, determines the shape of the extruded products. The barrel is heated through the means of electrical or liquid-based (oil, steam) heaters. Besides the heat supplied by the barrel, there will be friction between the rotating screws and the wall of the barrel, generating a substantial amount of additional heat. Most commercial extruders have a modular design, i.e. interchangeable sections or choice of screw configurations. This allows configuration of the feed, transition and metering zones to be altered, which allows for the modification from standard to high shear extrusion.

The twin-screw extruder has two agitator assemblies mounted on parallel shafts (12). These shafts are driven through a splitter/reducer gearbox and rotate in the same direction (co-rotating) or in the opposite direction (counter-rotating). The screws are often intermeshing, which means that each agitator element interacts with both the surface of the corresponding element on the adjacent shaft, and the internal surfaces of the mixing chamber. The screws of the twin-screw extruder are modifiable by using transport elements (used to convey the product) and kneading elements (used to mix the product). These kneading elements can be placed in different angles, providing more or less mixing, and thus higher or lower shear forces. In general, co-rotating shafts have better mixing capacities as the surfaces of the screws move towards each other. Therefore, co-rotating twin-screw extruders are preferred to counter-rotating instruments when it comes to solid dispersions and mixing/dissolving drug into a polymeric carrier. Also the screw configuration is of importance for generating solid dispersions, since the mixing zone also determines the degree of mixing. Extrusion processing requires close monitoring and understanding of the various parameters such as temperature settings, feeding rate and screw speed. These parameters together determine the viscosity of the melt in the extruder and the shear rate created by the friction between screws and barrel wall, known as the torque of the machine, i.e. the resistance that the gearbox measures as a consequence of the viscosity in the extruder.

Numerous pharmaceutical applications of hot melt extrusion are described in the literature which has been extensively reviewed by Breitenbach (12). One such example is given by Verreck et al. and Six. et al., who describe hot melt extrusion of the poorly water soluble drug itraconazole with HPMC as the polymeric carrier (25-28). Both dissolution and bioavailability were significantly enhanced for this drug substance when melt extruded with HPMC in a drug/polymer ratio of 40/60 wt %.

For drugs like itraconazole, thermal degradation was not observed. However, a number of drugs exist which are thermo-labile. In these cases, hot melt extrusion is not applicable, unless a plasticizer is used which reduces the process temperature enough to allow for thermal treatment. Typically, these plasticizers are used in a concentration range of 5 to 30 wt % of the polymer content. This is a major disadvantage, since traditional plasticizers add to the final dosage weight, which may become unacceptably high in cases where the drug dosage is high. Therefore, it would be beneficial to have a material that lowers the processing temperature without being present in the final formulation. The use of a supercritical fluid which plasticizes the polymeric carrier, and upon release of the pressure, expands to a gas and escapes from the polymer to create a foam, will be investigated for this purpose.

4. Applications of supercritical fluids in hot melt extrusion.

Applications of supercritical fluids to hot melt extrusion are not new in the polymer science. In the last decade, research in this area has received increasing attention. The applications generally focus on the viscosity reduction and foam formation by the supercritical fluid.

Adding supercritical carbon dioxide to a polymer melt can lower the melt viscosity (29). This occurs through two mechanisms: first, the carbon dioxide absorbs between the polymer chains causing an increase of free volume and decrease of chain entanglement. Second, the carbon dioxide acts as a molecular lubricant that further reduces the melt viscosity.

Elkovitch et al. reported the viscosity reduction of polystyrene and poly(methyl methacrylate) (PMMA) by supercritical carbon dioxide when measured in a single screw extruder (30, 31). They reported a viscosity reduction of 70% for PMMA and 40-50% for polystyrene. Further, they investigated blends of PMMA and polystyrene in a twin screw melt extruder and observed that blending improved due to the reduced viscosity of the two polymers by injecting supercritical or subcritical carbon dioxide (31). Lee et al. investigated the viscosity reduction of polyethylene/polystyrene (PE/PS) blends and they also observed a significant plasticisation effect when measured in a twin screw extruder (32). They further investigated foam formation that was observed when the extruded polymer/gas mixture was exiting the die. It was found that pore size could be altered by changing pressure and carbon dioxide concentration. Other groups have also investigated foam formation upon exiting the die of a melt extruder. Park et al., for example, studied the continuous micro-cellular foam formation of polystyrene using a single screw extruder (33).

These examples demonstrate that injection of a supercritical fluid in both a single screw as well as a twin-screw extruder should be feasible. However, when using a twin-screw extruder, the use of an optimal screw design must be taken into account when optimising the process since (32): (a) At the injection port of the carbon dioxide the pressure fluctuations should be minimized to obtain a stable injection. Therefore, transport elements instead of kneading elements should be used at the site of injection. (b) Injected carbon dioxide should not be allowed to leak from upstream orifices, which is achieved by a melt seal using reversed elements. (c) The pressure downstream should be maintained at a sufficiently high level to ensure that the supercritical carbon dioxide remains dissolved in the polymer. This can be obtained by providing high die resistance. (d) Complete dissolution of carbon dioxide can be assured by using kneading elements to improve mixing downstream of the supercritical fluid introduction.

The experimental set up for a single screw extruder, has been proposed by Elkovitch et al. (30). Carbon dioxide is supplied from a gas cylinder, cooled to obtain liquid carbon dioxide, and pumped into the extruder using a syringe pump.

5. Pharmaceutical polymers

Polyvinylpyrrolidone-vinyl acetate 64 (PVP-VA 64) and Eudragit E100 PO were used as model polymers for the experiments.

PVP-VA 64 is manufactured by a free-radical polymerisation of 6 parts of vinylpyrrolidone and 4 parts of vinyl acetate in isopropanol. PVP-VA 64 is soluble in water as well as in a number of organic solvents, such as ethanol, isopropanol, butanol and methylene chloride. PVP-VA is an amorphous polymer with a glass transition around 103° C. Thermal decomposition starts above 225° C.

Eudragit E100 PO is a polymethacrylate made up of 2-dimethyl aminoethyl methacrylate, methyl methacrylate and n-butyl methacrylate. Eudragit E100 PO is soluble in acidic solutions up to pH 5 and swells in solutions above pH 5. The polymer is soluble in a number of organic solvents including isopropanol, acetone, methanol and ethanol. Thermal decomposition starts above 200° C. Eudragit E100 PO is an amorphous polymer with a glass transition around 50° C.

### II. SUMMARY OF THE INVENTION

The aim of the current research project was to explore and investigate the combined possibilities of supercritical fluids and hot melt extrusion of pharmaceutically acceptable polymers. The influence of injecting a pressurized gas as plasticizer for the polymer was investigated, as well the ability to form a foam upon expansion of the pressurized gas.

Given the importance of an optimal screw configuration, to be able to build up pressure in the extruder and dissolve the carbon dioxide into the polymeric carrier, the initial experiments focused on the extruder set up and optimisation of the screw configuration. For these experiments, PVP-VA 64 was used as a model polymer. Therefore, different screw configurations and extruder set ups were tested and evaluated.

Once a screw configuration and extruder set up were selected, the next step was then to evaluate the effect of the different parameter settings on the plasticising and foam formation capabilities of the carbon dioxide in the polymeric carrier. This was done for both PVP-VA 64 as well as Eudragit E100 PO.

Further, the physicochemical characteristics of the polymers before and after treatment with carbon dioxide were investigated.

Further still, the processing parameters during the extrusion process as well as the physicochemical properties of the melt extrudate of itraconazole/PVP-VA 64 10/90 w/w and 40/60 w/w were investigated as carbon dioxide was injected during extrusion.

The present invention relates to particles of the polymer PVP-VA-60 or the polymer Eudragit-E100-PO, characterized in that said particles are shaped as platelets. Platelets are minute flattened particles; i.e. particles of which the thickness is smaller than the length and width.

In particular, this invention concerns particles of the polymer PVP-VA-60 wherein the specific surface area is larger than 0.350 m²/g.

Additionally, this invention concerns particles of the polymer Eudragit-E100-PO wherein less than 40% (w/w) is smaller than 100 μ.

Further, this invention relates to particles comprising the polymer PVP-VA-60 or the polymer Eudragit-E100-PO, and an active ingredient, characterized in that said particles are shaped as platelets.

Specifically, this concerns particles wherein the active ingredient is itraconazole. More in particular, this relates to particles wherein the weight by weight ratio of itraconazole to polymer ranges from about 10/90 to about 40/60.

As described hereinafter, the particles according to the invention have improved compressibility (given by the equation tapped density—bulk density/tapped density); in particular, the compressibility is larger than 25%. In addition, the particles are easy to mill.

A further aspect of the present invention concerns pharmaceutical dosage forms comprising a therapeutically effective amount of particles as defined hereinbefore.

Also, the invention relates to a process of preparing such pharmaceutical dosage forms comprising the steps of intimately mixing particles as defined hereinbefore with pharmaceutically acceptable excipients and making from the thus obtained mixture pharmaceutical dosage forms comprising a therapeutically effective amount of particles.

Further, the invention relates to a process of preparing particles as defined hereinbefore comprising the steps of
feeding the polymer, or a mixture of the polymer and the active ingredient, into a melt extruder,
transporting the polymer, or a mixture of the polymer and the active ingredient, through the barrel of the melt extruder by means of a screw modified with transport elements and with kneading elements,
injecting pressurized gas into the barrel of the melt extruder through a port located in the barrel,
mixing the polymer, or a mixture of the polymer and the active ingredient, and the pressurized gas under subcritical or supercritical conditions
expanding the polymer, or a mixture of the polymer and the active ingredient, after the die plate, and
milling the extrudate,
characterized by creating a melt seal before the site of the pressurized gas injection by placing a reversing transport element in the screw configuration at said site.

### III. DETAILED DESCRIPTION OF THE INVENTION

1. Materials

Itraconazole was obtained from Janssen Pharmaceutica N.V. (purity>99%).

PVP-VA 64 was obtained from BASF (BASF, Ludwigshafen, Germany). The following lot numbers were used during the experiments: lot 10232285, 10237176 and 93875968-E0.

Eudragit E100 PO was obtained from Rhom (Rhom, Darmstadt, Germany). The following lot number was used during the experiments: lot 0410231047.

CO₂ (>99.9 vol %, purity 3.0) was supplied in gas cylinders with dip tube (Messer, Machelen, Belgium).

2. Methods

2.1. Melt extrusion

The melt extrusion trials were performed with a Leistritz Micro 18 co-rotating intermeshing twin screw extruder. The screw diameter was 18 mm and the length to diameter ratio (L/D) was 40, divided over 4 barrel segments of 5 L/D each and 1 barrel element of 20 L/D. The first barrel segment was water cooled only. This was done to prevent melting of the material at the feed port. This could cause blockage of the feed due to build up of material directly below the powder feeder. All other barrel elements were heated and cooled independently. At the end of the barrel, a flange and die plate were installed which were heated separately as well. Separate heating and cooling is advantageous to better control the temperature throughout the barrel. At each new condition, at least 10 minutes was allowed to pass to achieve equilibrium before the resulting torque, pressure, etc. were documented.

The pressure inside the barrel was measured at three locations: before and after the CO₂ injection port and in the flange. As mentioned already in I3. and I.4., extruder set up and screw configuration optimisation are extremely important. Therefore, the experiments were performed with different extruder set ups as schematically shown in FIGS. 1 and 2. Also several screw configurations were investigated (FIGS. 3 and 4).

### BRIEF DESCRIPTION OF THE FIGURES

FIG. 1: Schematic set up 1 of the twin screw extruder. Carbon dioxide is injected in zone 3 and a vent port is foreseen in zone 6 to expand the carbon dioxide back to atmospheric pressure.

FIG. 2: Schematic set up 2 of the twin screw extruder. Carbon dioxide is injected in zone 3. Further downstream, the barrel is completely closed. Carbon dioxide is released back to atmospheric pressure upon exiting the die.

FIG. 3: Schematic set up of screw configuration 1. One melt seal is obtained by a reversing transport element before carbon dioxide injection and another melt seal before the vent port. Between those two melt seals a transport zone and two mixing zones are provided.

FIG. 4: Schematic set up of screw configuration 2. One melt seal is obtained by a reversing transport element before carbon dioxide injection and another melt seal is obtained in the die plate. To distribute the carbon dioxide better in the polymer, two mixing zones are provided downstream after the melt seal.

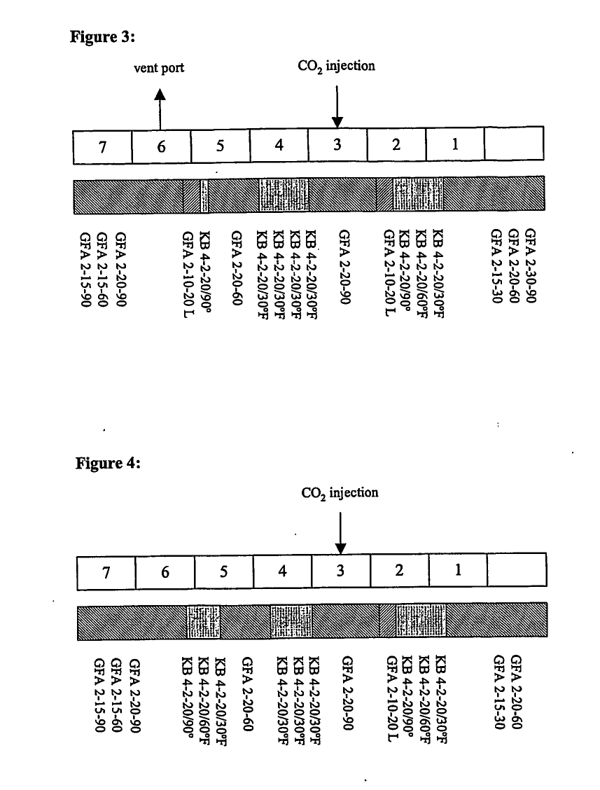

Polymer was fed with a K-Tron loss in weight feeder system (K-Tron, Switzerland). This gravimetric feeding system accurately feeds the polymer into the extruder. Multiple powder feeders are possible to meter the different materials individually. In the experiments, only one feeder was used since only pure polymer was extruded. The screw configuration, as shown in FIGS. 3 and 4, is usually described by the following terminology (34):
e.g. GFA-2-10-30: G=co-rotating, F=conveying, A=free meshing, 2=number of threads, 10=pitch, 30=length of screw element
e.g. KB 4-2-20/30° F.: KB=kneading block, 4=number of kneading elements, 2=number of threads, 20=length of kneading block, 30°=twisting angle of the individual kneading segments, F=conveying (L=back conveying)

2.1.1. Extrusion of PVP-VA 64 without CO₂ injection

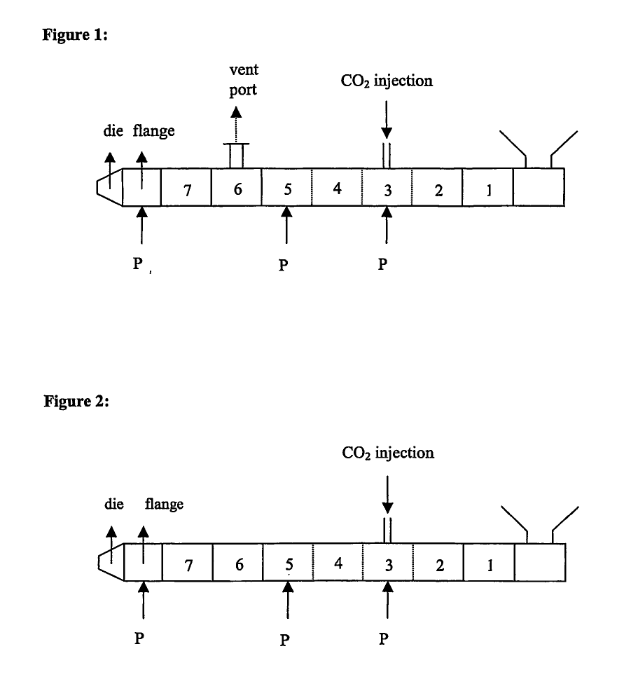

The first set of experiments with PVP-VA 64 without carbon dioxide injection was performed using extruder set up 1 and screw configuration 1 as illustrated in FIGS. 1 and 3. The parameter settings that were used are presented in Table 3 (see Results and Discussion Section). Experiments 1 to 4 were performed to investigate the influence of the screw speed by gradually increasing the speed from 50 rpm to 250 rpm.

Experiments 5 to 10 were performed to investigate the influence of the feeding rate within a range of 0.5 to 3 kg/hr. Finally, experiments 11 to 15 were performed to evaluate the influence of the temperature. Experiments 5 and 6 were performed in duplicate, all other experiments were performed once.

The next set of experiments was performed with a modified screw configuration and extruder set up as illustrated in FIGS. 2 and 4. The parameter settings are presented in Table 4 (see Results and Discussion Section). Again, experiments 1 to 4 were performed to investigate the influence of the screw speed, experiments 5 to 10 to investigate the influence of the feeding rate and experiments 11 to 15 were performed to evaluate the influence of the temperature. These experiments were performed in duplicate.

The third set of experiments without carbon dioxide injection was performed to determine the minimal processing temperature of the barrel keeping the first two zones at increased temperature, i.e. at 180° C. (zone 1 and 2 in FIG. 2), while gradually decreasing all other zones. The feeding rate (1 kg/hr) and screw speed (100 rpm) were kept constant throughout the experiments. The experiments were performed with a screw configuration and extruder set up as illustrated in FIGS. 2 and 4. The parameter settings are presented in Table 5 (see Results and Discussion Section). The experiments were performed once.

2.1.2. Extrusion of PVP-VA 64 with CO₂ injection

Carbon dioxide was pressurized and injected in the extruder using an ISCO 260D syringe pump (ISCO, US). CO₂ was provided as liquid (T=20° C.; P=56 bar) from a gas cylinder with a dip tube and cooled to 1.5° C. with a spiral tube in a cooling bath (Analis Heto, CBN 8-30, Denmark). Cooling medium was a mixture of isopropanol/water 50/50 v/v. Also the cylinder of the pump was cooled to 1.5° C. The syringe pump can be operated in two metering modes, constant pressure rate (CPR) or constant flow rate (CFR). CPR has the advantage that a certain pressure can be delivered towards the melt extruder. On the other hand, CFR has the advantage that the amount of carbon dioxide injected in the extruder is exactly known. The experiments were performed both at CPR as well as at CFR to investigate whether there was a difference between both metering modes. Carbon dioxide was injected in the barrel through an injection nozzle located in barrel segment 3.

The initial screw configuration and extruder set up are shown in FIGS. 1 and 3. The first set of experiments were performed to evaluate the influence of injecting carbon dioxide at CPR between 20 to 50 bar. The temperature settings were maintained at 180° C. (all zones), the screw speed between 100 and 150 rpm and the feeding rate of 1 kg/hr. These experiments were performed in duplicate.

In a second set of experiments with injection of carbon dioxide, a modified screw configuration and extruder set up were applied as illustrated in FIGS. 2 and 4. The injection of pressurized CO₂ was again performed at CPR between 30 to 55 bar, at a temperature setting of 180° C. (all zones), a screw speed of 150 rpm and a feeding rate of 1 kg/hr. These experiments were performed in duplicate.

The third set of experiments with carbon dioxide injection was also performed using the screw configuration and extruder set up as illustrated in FIGS. 2 and 4. The temperature settings were maintained at 180° C. for zone 1 and 2, while temperature settings of all other zones were gradually decreased between 180° C. and 120° C. to find the minimal processing temperature under carbon dioxide injection. Carbon dioxide was injected at CPR between 35 and 55 bar. The screw speed and feeding rate were kept constant at 100 rpm and 1 kg/hr respectively. The parameter settings are shown in Table 6 (see Results and Discussion Section). The experiments were performed in duplicate.

The next set of experiments was performed to evaluate the influence of the different parameter settings (feeding rate, screw speed and carbon dioxide pressure) on the torque, polymer foaming, pressure in the extruder, and other factors. The experiments were performed using the screw configuration and extruder set up as illustrated in FIGS. 2 and 4. Table 7 (see Results and Discussion Section) shows the parameter settings at 140° C. (zone 1 and 2 were maintained at 180° C.). Experiments 1-3 were performed to gradually decrease the temperature settings to reach a condition of steady state in the extruder while injecting carbon dioxide. From steady state, experiments 4-7 were performed to investigate the influence of the carbon dioxide pressure between 35 and 50 bar, experiments 8-10 were performed to investigate the influence of the screw speed between 100 and 200 rpm and experiments 11-16 to investigate the feeding rate between 0.5 and 1.5 kg/hr. These experiments were performed once. Similar experiments were performed at temperature settings of 130° C., whereby carbon dioxide was injected at CPR between 35 and 60 bar, and at 120° C. with injection pressures between 60 and 75 bar. The other parameters, screw speed and feeding rate, were kept constant at 100 rpm and 1 kg/hr, respectively. These experiments were performed once.

A final set of experiments was performed to evaluate the effect of injecting CO₂ under CFR instead of CPR. The experiments were performed using the screw configuration and extruder set up as illustrated in FIGS. 2 and 4. Table 8 shows the parameter settings at 140° C. and Table 9 at 130° C. (zone 1 and 2 were kept constant at 180° C.) (see Results and Discussion Section). Carbon dioxide was injected at CFR varying between 0.5 and 30 ml/min. The screw speed and feeding rate were maintained at 100 rpm and 1 kg/hr, respectively. The experiments were performed once.

To prepare a sample for physicochemical characterization of the polymer after carbon dioxide treatment, the following conditions were used: the temperature was set at 140° C. (except zone 1 and 2, which were kept at 180° C.), the feeding rate at 1 kg/hr and the screw speed at 100 rpm.

2.1.3 Extrusion of (a) itraconazole/PVP-VA 64 10/90 without CO₂ injection and (b) itraconazole/PVP-VA 64 40/60 without CO₂ injection

The experiments without carbon dioxide injection were performed to investigate the influence of temperature settings on torque of the machine. The parameter settings are presented in Table 13 and 19 (see Results and Discussion Section).

Experiments 1a to 4a and 1b to 5b were performed to evaluate the influence of the temperature, whereby the temperature was gradually decreased for all zones.

Experiments 5a to 8a and 6b to 10b were performed to investigate the influence of temperature, with the first two zones maintained at 180° C. All these experiments were performed at least in duplicate.

2.1.4. Extrusion of (a) itraconazole/PVP-VA 64 10/90 with CO₂ injection and (b) itraconazole/PVP-VA 64 40/60 with CO₂ injection

Carbon dioxide was pressurized and injected in the extruder using an ISCO 260D syringe pump (ISCO, US). CO₂ was provided as liquid (T=20° C. ; P=56 bar) from a gas cylinder with a dip tube and cooled to 1.5° C. with a spiral tube in a cooling bath (Analis Heto, CBN 8-30, Denmark). Cooling medium was a mixture of isopropanol/water 50/50 v/v. Also the cylinder of the pump was cooled to 1.5° C. The experiments were performed at CPR. Carbon dioxide was injected in the barrel through an injection nozzle located in barrel segment 3.

The first set of experiments was performed to investigate the effect of injecting carbon dioxide on the torque of the extruder. All temperature zones were gradually decreased between 180° C. and 140° C., respectively between 160° C. and 125° C. to find the minimal processing temperature. Carbon dioxide was injected at CPR between 35 and 40 bar.

The screw speed and feeding rate were kept constant at 100 rpm and 1 kg/hr, respectively. A further set of experiments was performed with the first two zones at increased temperature (180° C.). The parameter settings are shown in Table 14 and 15 (see Results and Discussion Section). The experiments were performed in duplicate.

2.1.5. Extrusion of Eudragit E100 PO without CO₂ injection

All experiments with Eudragit E100 PO were performed using the screw configuration and extruder set up as illustrated in FIGS. 2 and 4. This screw configuration and extruder set up were selected based on the previous experiments performed using PVP-VA 64.

The experiments with Eudragit E100 PO without carbon dioxide injection were performed to investigate influences of temperature settings, feeding rate and screw speed on the torque of the machine. The parameter settings are presented in Table 26 (see Results and Discussion Section).

Experiments 1 to 5 were performed to investigate the influence of the screw speed within a range of 50 to 250 rpm and experiments 6 to 10 to investigate the influence of the feeding rate within a range of 0.5 to 2.5 kg/hr. Experiments 11 to 14 were performed to evaluate the influence of the temperature, whereby the temperature is gradually decreased for all zones. On the other hand, experiments 15 to 20 were performed to investigate the influence of temperature, with the first two zones maintained at 180° C.

All these experiments were performed in duplicate.

2.1.4. Extrusion of Eudragit E100 PO with CO₂ injection

Carbon dioxide was injected in the extruder as described in Section 2.1.2.

The first set of experiments with carbon dioxide injection for Eudragit E100 PO was performed to investigate the effect of injecting carbon dioxide on the torque of the extruder. The temperature settings were kept constant at 180° C. for zone 1 and 2, while temperature settings of all other zones were gradually decreased between 180° C. and 110° C. to find the minimal processing temperature. Carbon dioxide was injected at CPR between 20 and 45 bar. The screw speed and feeding rate were kept constant at 100 rpm and 1 kg/hr, respectively. The parameter settings are shown in Table 27 (see Results and Discussion Section). The experiments were performed in duplicate.

The next set of experiments was performed to evaluate the influence of the different parameter settings (feeding rate, screw speed and carbon dioxide pressure) on the torque, polymer foaming, pressure in the extruder, and other factors. Table 28 (see Results and Discussion Section) shows the parameter settings at 120° C. (zone 1 and 2 were kept constant at 180° C.). Experiments 1-6 were performed to investigate the influence of the carbon dioxide pressure between 35 and 70 bar, experiments 7-9 were performed to investigate the influence of the screw speed between 100 and 200 rpm and experiments 10-12 to investigate the feeding rate between 0.5 and 1.5 kg/hr. These experiments were performed once.

A final set of experiments was performed to evaluate the effect of injecting CO₂ under CFR instead of CPR. Table 29 shows the parameter settings at 120° C. (zone 1 and 2 were kept constant at 180° C.). Carbon dioxide was injected at CFR varying between 0.5 and 15 ml/min. The screw speed and feeding rate were kept constant at 100 rpm and 1 kg/hr respectively. The experiments were performed once.

To prepare a sample for physicochemical characterization of the polymer after carbon dioxide treatment, following conditions were used: the temperature was set at 120° C. (except zone 1 and 2, which were kept at 180° C.), a feeding rate of 1 kg/hr and a screw speed of 100 rpm.

2.2. Milling

Prior to analysis, the polymer samples processed with pressurized carbon dioxide, were milled using a Bamix lab scale mill (Bamix, Mettlen, Switzerland). PVP-VA 64 was milled for 30 seconds and the fraction below 500 μm (ASTM E11-61:35 mesh/inch) was retained for further characterization.

Eudragit E100 PO was also milled for 30 seconds and a fraction below 250 μm (ASTM E11-61:60 mesh/inch) was retained for further analysis.

Itraconazole/PVP-VA 64 10/90 and 40/60 were also milled for 30 seconds and a fraction below 250 μm (ASTM E11-61: 60 mesh/inch) was retained for further analysis.

2.3. Modulated Differential scanning calorimetry

Modulated Differential scanning calorimetry (MDSC) was performed to evaluate the thermal characteristics before and after melt extrusion with injection of pressurized carbon dioxide. Both polymers are amorphous and thus they possess a glass transition, which is the transition between regions of high and low molecular mobility. A glass transition is observed as an inflection in the DSC profile when heat flow is plotted against temperature. Often, the glass transition is accompanied with an enthalpy relaxation, which is seen as an endothermic signal superimposed on the glass transition. Furthermore, these polymers can be hydrophilic and thus they may have adsorbed a significant amount of water. Evaporation of water is also an endothermic process. These signals make interpretation of the glass transition difficult. Therefore, MDSC was used so that the different thermal events could be elucidated with regard to the reversible and non-reversible transitions. A glass transition will be observed as a reversing signal, while enthalpy relaxation and solvent evaporation are seen in the non-reversing signal.

The measurements were performed using a TA Instruments modulated DSC Q1000 differential scanning calorimeter and thermal analysis controller (TA Instruments, New Castle, Del., USA). Cooling was provided with a TA Instruments refrigerated cooling system (RCS, TA Instruments). Data were treated mathematically using the resident TA Q-series software. Calibration was carried out using indium (5° C./min, Tₘ=157.92° C., cell constant=1.0783) and sapphire (Cp constant=1.093) as reference materials. The samples were analysed in standard (open) aluminum as well as in hermetically sealed TA Instruments pans. Nitrogen was used as the purge gas at 50 ml/min. The glass transition was measured at the inflection point as half the height of the shift in the heat flow signal (T_{g 1/2 cp}).

The TA Instruments DSC Q1000 uses a newly designed principle that is a hybrid between heat flux and power compensation modes of operation (35). By considering thermal resistance and heat capacity imbalances, and by including a heating rate difference between sample and reference, the baseline and resolution are significantly improved.

Approximately 2-6 mg of the polymer was heated from 0° C. to 140° C. with a heating rate of 2° C./min, a period of 60 s and an amplitude of ±0.32° C. The temperature limits were selected such that the whole glass transition region was covered for both polymers. The modulation parameters were selected because they are used routinely within our laboratory.

The samples measured in open pans as well as in hermetically sealed pans were analysed at least in duplicate.

2.4. Thermogravimetric analysis

Thermogravimetric analysis (TGA) was performed to measure residual solvent before and after treatment with carbon dioxide in the melt extruder. As mentioned previously, extraction (of solvents, impurities, monomers) is a possible industrial application of sub- or supercritical fluids. In other words, TGA is performed to evaluate whether solvent or weight loss from the sample occurred after treatment with carbon dioxide. During TGA, the mass of a sample is accurately measured while the temperature is heated at a constant rate. This method is a quantitative, not qualitative, measurement for solvent loss, since it is not known which solvents evaporate.

The samples were measured with a TA Instruments Hi-Res TGA 2950 (TA instruments, New Castle, Del., USA) equipped with data station TA2100. Approximately 10 mg of sample was weighed in an aluminum pan of 30 microliter volume and heated from room temperature at a heating rate of 20° C./min. The endpoint was set at 300° C. or at a weight loss of 20%. The samples were measured at least in duplicate.

2.5. Specific surface area

The specific surface area was measured to evaluate wether the morphology of the polymer was changed after treatment with carbon dioxide. When the polymer exits the die, the dissolved carbon dioxide is converted into the gaseous phase and escapes from the polymer matrix. This creates a foam and thus morphology may have changed (32, 33).

The specific surface area can be defined as the total surface area per unit weight (volume) of the powder. Surface area measurement is usually carried out by either gas permeability or adsorption.

Gas adsorption is determined by placing a sample of the powder in the sample holder and removing the air within (degassing). After degassing, known volumes of an adsorbing gas, usually nitrogen, are introduced. From the knowledge of pressure and temperature before and after introduction of the adsorbing gas, calculations of total sample surface area can be made. The amount of gas or liquid adsorbed to a sample as a monolayer is directly proportional to the specific surface area of the sample. The relationship between the volume of a gas adsorbed by a powder and the equilibrium pressure of the gas surrounding it at constant pressure, leads to typical adsorption isotherms. The most widely used calculation technique is based on the BET theory by Brunauer, Emett and Teller (36).

The specific surface area was measured with a Quantachrome (Quantachrome, Greenvale, N.Y., USA) using Kr/He gas mixtures (0.1, 0.2 and 0.3 mole fraction) at 1.5 bar and 25 ml/min flow rate. Adsorption time was between 20 and 30 minutes. Calibration was done with a known quantity of Kr. Samples were degassed repeatedly (6 times) prior to analysis by adsorbing and desorbing using a constant flow of the Kr/He 0.3 mole fraction gas mixture.

2.6. Particle size

Since the treated polymer was milled prior to analysis (Section 2.2.), the particle size was measured as a comparison with the untreated polymer. Particle size is important, especially when comparing dissolution data.

The particle size and particle size distribution was measured by the vibrating sieve method. Using this method, a set of sieves with known mesh size (respectively 75, 150, 250, 500, 850 and 1000 micron) and known tar weight, was placed on top of each other and a known amount of the powder was poured on the top sieve. The whole stack was placed on a vibrating plate for 10 minutes at an amplitude of 1.5 mm after which each sieve was weighed to obtain a particle size average and distribution.

The analysis was performed once for each sample. The particle size of PVP-VA 64 before and after treatment was measured with 10 g of material. The particle size of Eudragit E100 PO before treatment was measured on 50 g, while after treatment the analysis was done on 10 g of material.

2.7. Dissolution

Since morphology could have changed due to the foam formation at the exit of the extruder, dissolution properties could have changed as well.

Therefore, the dissolution of PVP-VA 64 was measured by adding 10 g of a sample to 500 mL purified water (37° C.), while stirring with a paddle at 50 rpm (USP II apparatus). The dissolution was followed for 1 hour with samples of the dissolution medium taken after 5, 15, 30, 45 and 60 minutes. An aliquot of 3 ml was filtered through a Millex HV 0.45 μm filter (Millipore SLHV R04 NL) and diluted with purified water. The sample was not replaced with fresh solvent. The concentration of PVP-VA 64 was measured photometrically by the formation of the iodine complex (37). Therefore, 5 mL of the diluted sample solution was mixed with 2.5 mL of 0.2 M citric acid solution and 1 mL of 0.006 N iodine solution (0.81 g of freshly sublimed iodine and 1.44 g of potassium iodide dissolved in 1000 mL of water). The absorbance was measured by UV at 470 nm after exactly 30 minutes. The experiments were performed in triplicate.

Dissolution of Eudragit E100 PO was measured by adding 10 g of sample to 900 mL 0.1 N HCl (37° C.), while stirring with a paddle at 50 rpm (USP II apparatus). The dissolution was followed for 1 hour with samples of the dissolution medium taken after 5, 15, 30, 45 and 60 minutes. An aliquot of 3 ml was filtered through a Millex HV 0.45 μm filter (Millipore SLHV R04 NL). The sample was not replaced with fresh solvent. The concentration of Eudragit E100 PO was measured gravimetrically. Therefore, 2 ml of the filtered sample was transferred into a petri dish and placed in the vacuum oven at respectively 60° C. for 2 hours, 50° C. for 20 hours and at 100° C. for 2 hours. The weight of the petri dishes is measured after respectively 2, 22 and 24 hours until a constant weight is obtained. The experiments were performed in triplicate.

Alternatively, the dissolution of Eudragit E100 PO was also measured by adding 1 g of sample to 900 ml 0.01 N HCl using the same procedure as described above.

Dissolution testing of itraconazole/PVP-VA 64 10/90 and 40/60 was performed on milled melt extrudate samples and compared with physical mixture containing crystalline itraconazole. Samples with a 200 mg dose were directly added to 500 ml of simulated gastric fluid without pepsine (SGF) at 37° C. The dissolution was assessed using a paddle rotating at 100 rpm (USP II apparatus). The release was followed for 1 hour and samples were taken after 5, 15, 30, 45 and 60 minutes. An aliquot of 3 ml was filtered through a Milex HV 0.45 mm filter (Millipore SLHV R04NL). The sample was not replaced by fresh solvent. The concentration of itraconazole was quantified with UV at the maximum wavelength of 254 nm.

2.8. Light microscopy

Light microscopy was performed to evaluate the morphology of the polymer after treatment with carbon dioxide, but before milling. A thin film of the unmilled sample was placed on the slide glass and measured directly. A Nikon Eclipse E600 polarised light microscope was used for these studies. Photographs were taken at a lens magnification of 4.

2.9. Scanning electron microscopy

Scanning electron microscopy was performed to evaluate the morphology of the milled polymer before and after treatment. The milled sample was afixed on the stub with adhesive tape. The mounted samples were coated with a layer of gold using a Balzers sputtering device. Samples were placed in a multiple specimen holder of the scanning electron microscope. The samples were observed with a JEOL JSM-5510 (Japan Electron Optics Laboratory LTD) scanning electron microscope and were scanned at 10-20 kV. Digital images were processed in Adobe Photoshop.

2.10. Melt viscosity

The melt viscosity of the polymers was measured to evaluate whether shear thickening or shear thinning behaviour occurred and to investigate the viscosity behaviour as a function of temperature.

The melt viscosity was measured with a Rheometrics RDA-II rheometer using parallel plates in dynamic strain frequency sweep method (Rheometrics, Piscataway, N.J., US). PVP-VA 64 was measured between 140° C. and 180° C. using frequencies starting at 0.1 rad/s and ending at 100 rad/s. The diameter of the plates was 40 mm and the gap between the parallel plates was 1 mm. Experiments were performed in duplicate. Eudragit E100 PO was measured between 100° C. and 150° C. at frequencies starting at 1 rad/s and ending at 100 rad/s. The diameter of the plates was 25 mm and the gap between the parallel plates was 1.5 mm. Experiments were performed in duplicate.

2.11. Bulk and tapped volume

The powder bulk and tapped volume were measured using a jolting volumeter (J. Engelsmann A. G. Ludwigshafen am Rhein, Germany). A known amount of the sample before and after treatment was poured into a graduated cylinder and the volume of the powder bed was measured to obtain the bulk volume. Then the volumetric flask was transferred into the tapping apparatus and tapped 500 times. The volume of the powder bed was measured again to obtain the tapped volume.

2.12. Micro Attenuated Total Reflectance (microATR)

MicroATR was performed with a Nicolet Magna 560 FTIR spectrophotometer equipped with a DTGS/KBr detector and Ge/KBr beamsplitter. 32 Scans were taken within the wavelength range of 4000 cm⁻¹ to 400 cm⁻¹ at a resolution of 1 cm⁻¹. Samples were measured using a Harrick Split Pea/Si crystal microATR accessory.

### IV. RESULTS AND DISCUSSION

1. PVP-VA 64

PVP-VA 64 was used as one of the model polymers for the melt extrusion experiments with injection of carbon dioxide. The first part of the experiments focused on the extruder set up and screw configuration to allow for the injection of pressurized CO₂ and the build up of pressure within the extruder. The experiments were first performed without carbon dioxide injection, to learn more on the extrusion behaviour of PVP-VA 64. After these experiments, trials with CO₂ injection were performed and the physico-chemical characteristics of PVP-VA 64 before and after treatment were assessed. Finally, similar experiments were set up with physical mixtures of itraconazole/ PVP-VA 64 10/90 and 40/60.

1.1. Melt extrusion of PVP-VA 64 without CO₂ injection

The first experiments were performed to investigate the extrusion behaviour of PVP-VA 64 with the Leistritz Micro 18 co-rotating twin-screw extruder, without CO₂ injection. Each set of parameters for temperature, screw speed and feeding rate resulted in a specific viscosity of the polymer in the twin-screw extruder which was reflected by the torque of the machine. When the viscosity of the polymer in the extruder became too high, the torque reached a value above 100% and the machine shut down automatically. Therefore, the purpose of these experiments was to find the minimal temperature settings at which maximum torque was reached for a given feeding rate and screw speed. This allowed an evaluation of the effect of CO₂ on the torque for future experiments when injecting the pressurized gas.

Experiments with PVP-VA 64 without carbon dioxide injection were performed first using extruder set up 1 and screw configuration 1 as illustrated in FIGS. 1 and 3. This set up was chosen with an eye towards injecting the pressurized gas at barrel element 3, providing intimate contact between polymer and CO₂ in the mixing zone (barrel element 4), and extracting the carbon dioxide at element 6 through the vent port. Pressure was then expected to build up between zone 3 and 6 through the use of reversed transport elements which created a polymer melt seal between element and barrel. The parameter settings that were used and the resulting torque are presented in Table 3. Maximum torque was reached at 160° C. with a feeding rate of 1 kg/hr and a screw speed of 100 rpm (experiments 11-15 in Table 3). This means that further decreasing the temperature resulted in automatic shut down of the machine, and thus the minimal working temperature for PVP-VA 64 was 160° C.

It was also observed that an increase of the feeding rate caused an increase of the torque of the machine (experiments 5-10 in Table 3). This was expected, since at increased feeding rate, the same heat is consumed by more material per time unit. This created an increase of the melt viscosity and thus an increase in the torque.

One would also expect that an increase of the screw speed (i.e. more frictional energy created inside the barrel), decreased the viscosity and thus the torque. However, this was not observed in experiments 1 to 4 (Table 3). One possible explanation could be that PVP-VA 64 shows (non-Newtonian) shear thickening behaviour. Therefore the melt viscosity was measured as a function of shear rate (frequency in rad/s) and temperature. These profiles indicated that the apparent viscosity decreased with increasing shear rate and thus, it could be concluded that PVP-VA 64 does not show shear thickening behaviour. This meant that the reason for the increase of the torque with increasing screw speed had other causes and should be further investigated. In addition, it is clear that the viscosity increased with decreasing temperature, which confirmed the observations made during the extrusion trials.

The next set of experiments was performed with a modified screw configuration and extruder set up as illustrated in FIGS. 2 and 4. In contrast with the previous set up, extraction of carbon dioxide was now performed at the outlet of the extruder. The injection of the pressurized gas was still done at barrel element 3. Pressure was then expected to build up between a melt seal at zone 3 (created by a reversed transport element) and a melt seal obtained in the die opening when polymer was purged through that hole. Using this screw configuration, only one reversed transport element was used. The parameter settings and resulting torque are presented in Table 4. From these experiments we can observe that similar results were obtained using this screw configuration compared with that mentioned above, i.e. the effect of screw speed, feeding rate and temperature settings resulted in comparable values for the torque of the machine. Based on these experiments, it was observed that a left turning transport element creates significant torque. Alternatively, reversed paddle elements could be used to provide a melt seal instead of left turning transport elements. These paddle elements generally create less torque compared to transport elements (38). However, these elements are not available for the moment.

The third set of experiments was performed to determine the minimal processing temperature keeping the first two zones at increased temperature. The parameter settings and resulting torque are presented in Table 5. From these experiments, we observed that a maximum torque was reached below 150° C. when a feeding rate of 1 kg/hr and a screw speed of 100 rpm was used. Comparison with experiments 12 to 15 in Table 4, clearly shows that a constant higher temperature of 180° C. in zone 1 and 2, decreased the torque. Therefore it was possible to work below 160° C., without reaching maximum torque.

All further experiments for PVP-VA 64 were performed at a screw speed of 100 rpm, a feeding rate of 1 kg/hr and temperature settings of 180° C. for zones 1 and 2.

TABLE 3
Parameter settings for extrusion experiments of PVP-VA 64 without CO₂ injection - screw configuration 1 and extruder set
up 1. Investigation of the effect of screw speed, feeding rate and temperature settings on the torque of the extruder.

| | T1 | T2 | T3 | T4 | T5 | T6 | T7 | Tflange | Tdie | P1 | P2 | P3 | Tm | n | F | T |
|---|---|---|---|---|---|---|---|---|---|---|---|---|---|---|---|---|
| Nr. | (° C.) | (° C.) | (° C.) | (° C.) | (° C.) | (° C.) | (° C.) | (° C.) | (° C.) | (bar) | (bar) | (bar) | (° C.) | (rpm) | (kg/hr) | (%) |
| 1 | 150 | 170 | 170 | 180 | 180 | 180 | 180 | 180 | 180 | 2 | 9 | 15 | 187 | 50 | 0.5 | 33-40 |
| 2 | 150 | 170 | 170 | 180 | 180 | 180 | 180 | 180 | 180 | 2 | 11 | 13 | 187 | 100 | 0.5 | 43-55 |
| 3 | 150 | 170 | 170 | 180 | 180 | 180 | 180 | 180 | 180 | 2 | 12 | 12 | 188 | 150 | 0.5 | 48-63 |
| 4 | 150 | 170 | 170 | 180 | 180 | 180 | 180 | 180 | 180 | 2 | 13 | 10 | 188 | 250 | 0.5 | 51-71 |
| 5 | 180 | 180 | 180 | 190 | 190 | 190 | 190 | 190 | 190 | 2 | 9 | 10 | 197 | 150 | 0.5 | 37-49 |
| 6 | 180 | 180 | 180 | 190 | 190 | 190 | 190 | 190 | 190 | 2 | 9 | 13 | 197 | 150 | 1.0 | 39-51 |
| 7 | 180 | 180 | 180 | 190 | 190 | 190 | 190 | 190 | 190 | 2 | 9 | 17 | 197 | 150 | 1.5 | 42-54 |
| 8 | 180 | 180 | 180 | 190 | 190 | 190 | 190 | 190 | 190 | 2 | 10 | 20 | 197 | 150 | 2.0 | 45-58 |
| 9 | 180 | 180 | 180 | 190 | 190 | 190 | 190 | 190 | 190 | 2 | 10 | 21 | 197 | 150 | 2.5 | 45-59 |
| 10 | 180 | 180 | 180 | 190 | 190 | 190 | 190 | 190 | 190 | 2 | 11 | 22 | 197 | 150 | 3.0 | 46-60 |
| 11 | 180 | 180 | 180 | 190 | 190 | 190 | 190 | 190 | 190 | 2 | 9 | 13 | 197 | 150 | 1.0 | 39-52 |
| 12 | 170 | 170 | 170 | 180 | 180 | 180 | 180 | 180 | 180 | 3 | 10 | 17 | 186 | 150 | 1.0 | 45-58 |
| 13 | 170 | 170 | 170 | 170 | 170 | 170 | 170 | 170 | 170 | 3 | 12 | 22 | 175 | 150 | 1.0 | 51-65 |
| 14 | 160 | 160 | 160 | 160 | 160 | 160 | 160 | 160 | 160 | 2 | 19 | 34 | 165 | 150 | 1.0 | 70-88 |
| 15 | 160 | 160 | 160 | 160 | 160 | 160 | 160 | 160 | 160 | 2 | 31 | 35 | 164 | 100 | 1.0 | 84-100 |

TABLE 4
Parameter settings for extrusion experiments of PVP-VA 64 without CO₂ injection - screw configuration 2 and extruder set
up 2. Investigation of the effect of screw speed, feeding rate and temperature settings on the torque of the extruder.

| | T₁ | T₂ | T₃ | T₄ | T₅ | T₆ | T₇ | T_{flange} | T_{die} | P₁ | P₂ | P₃ | Tₘ | n | F | T |
|---|---|---|---|---|---|---|---|---|---|---|---|---|---|---|---|---|
| Nr. | (° C.) | (° C.) | (° C.) | (° C.) | (° C.) | (° C.) | (° C.) | (° C.) | (° C.) | (bar) | (bar) | (bar) | (° C.) | (rpm) | (kg/hr) | (%) |
| 1 | 150 | 170 | 170 | 180 | 180 | 180 | 180 | 180 | 180 | 2 | 1 | 11 | 186 | 50 | 0.5 | 54-65 |
| 2 | 150 | 170 | 170 | 180 | 180 | 180 | 180 | 180 | 180 | 2 | 1 | 10 | 187 | 100 | 0.5 | 52-65 |
| 3 | 150 | 170 | 170 | 180 | 180 | 180 | 180 | 180 | 180 | 2 | 1 | 9 | 187 | 150 | 0.5 | 57-72 |
| 4 | 150 | 170 | 170 | 180 | 180 | 180 | 180 | 180 | 180 | 2 | 1 | 8 | 188 | 250 | 0.5 | 52-72 |
| 5 | 180 | 180 | 180 | 190 | 190 | 190 | 190 | 190 | 190 | 2 | 1 | 8 | 197 | 150 | 0.5 | 35-46 |
| 6 | 180 | 180 | 180 | 190 | 190 | 190 | 190 | 190 | 190 | 2 | 1 | 10 | 197 | 150 | 1.0 | 37-48 |
| 7 | 180 | 180 | 180 | 190 | 190 | 190 | 190 | 190 | 190 | 2 | 1 | 13 | 197 | 150 | 1.5 | 42-54 |
| 8 | 180 | 180 | 180 | 190 | 190 | 190 | 190 | 190 | 190 | 2 | 1 | 15 | 197 | 150 | 2.0 | 48-60 |
| 9 | 180 | 180 | 180 | 190 | 190 | 190 | 190 | 190 | 190 | 2 | 1 | 15 | 196 | 150 | 2.5 | 50-62 |
| 10 | 180 | 180 | 180 | 190 | 190 | 190 | 190 | 190 | 190 | 2 | 1 | 16 | 196 | 150 | 3.0 | 51-63 |
| 11 | 180 | 180 | 180 | 190 | 190 | 190 | 190 | 190 | 190 | 2 | 1 | 11 | 197 | 150 | 1.0 | 39-50 |
| 12 | 170 | 170 | 170 | 180 | 180 | 180 | 180 | 180 | 180 | 2 | 1 | 15 | 184 | 150 | 1.0 | 52-64 |
| 13 | 170 | 170 | 170 | 170 | 170 | 170 | 170 | 170 | 170 | 2 | 1 | 18 | 176 | 150 | 1.0 | 59-73 |
| 14 | 160 | 160 | 160 | 160 | 160 | 160 | 160 | 160 | 160 | 2 | 1 | 27 | 165 | 150 | 1.0 | 83-100 |
| 15 | 160 | 160 | 160 | 160 | 160 | 160 | 160 | 160 | 160 | 2 | 1 | 33 | 164 | 100 | 1.0 | 85-100 |

TABLE 5
Parameter settings for extrusion experiments of PVP-VA 64 without CO₂ injection -
screw configuration 2 and extruder set up 2. Investigation of the effect
of the temperature settings on the torque of the extruder.

| | T₁-T₂ | T₃-T_{die}¹ | P₁ | P₂ | P₃ | Tₘ | N | F | T |
|---|---|---|---|---|---|---|---|---|---|
| Nr. | (° C.) | (° C.) | (bar) | (bar) | (bar) | (° C.) | (rpm) | (kg/h) | (%) |
| 1 | 180 | 180 | 3 | 1 | 12 | 186 | 100 | 1 | 31-41 |
| 2 | 180 | 170 | 3 | 2 | 15 | 174 | 100 | 1 | 35-46 |
| 3 | 180 | 160 | 2 | 1 | 28 | 165 | 100 | 1 | 50-63 |
| 4 | 180 | 150 | 2 | 1 | 44 | 157 | 100 | 1 | 70-82 |
| 5 | 180 | 140 | 2 | 1 | 55 | 153 | 100 | 1 | >100 |

¹T₃-T_{die}: all zones between T₃ and T_{die} were kept at the same temperature.

1.2. Melt extrusion of PVP-VA 64 with CO₂ injection

For the experiments with injection of a pressurized gas, the screw configuration was a very important parameter (32). This was especially through with an intermeshing, co-rotating twin-screw extruder, which was, by design, never completely filled with polymer throughout the length of the barrel. In other words, the motor drive would never be able to process a completely filled co-rotating twin screw extruder, since the motor power necessary to start rotating the screws would become too large for the gear box. This became obvious from the previous results (Section IV) where only one reversed transport element caused a large effect on the torque (and whereby only a short length of the barrel was completely filled with product at the left turning transport element).

To be able to build up pressure inside the barrel, the screw configuration had to be designed in such a way that a polymer melt seal was created (32). These polymer melt seals could be obtained by reversing transport elements, which were able to build up molten polymer locally between the reversing transport element and the barrel and as such provide for a melt seal.

The screw configuration and extruder set up that were used first are shown in FIGS. 1 and 3. As mentioned before, this set up was chosen with the idea to inject the pressurized gas at barrel element 3, mix the polymer with carbon dioxide in the mixing zone (barrel element 4), and extract the gas at element 6 through the vent port. Pressure was then expected to build up between zone 3 and 6 through the formation of molten polymer seals in these zones. Experiments with injection of pressurized CO₂ were performed at CPR between 20 to 50 bar (data not shown). It was possible to build up pressure inside the barrel between the two melt seals but neither a decrease in torque nor foaming of the polymer at the outlet of the extruder was observed. At the vent port, expansion of carbon dioxide was apparent. It was assumed that no foaming of the polymer was observed since all CO₂ was released at the vent port. A decrease in torque was not observed, probably because of the presence of the two reversing transport elements which create significant resistance to flow of the polymer through the barrel.

In other words, this screw configuration and extruder set up allowed pressure build-up inside the extruder, but did not provide for a measurable plasticising effect of the pressurized gas.

Therefore, a modified screw configuration and extruder set up were proposed as illustrated in FIGS. 2 and 4. Using this set up, it was possible to build up pressure inside the barrel to allow for polymer foaming. However, pressure build up was not constant and product was expelled from the machine with pressure release as a consequence. This phenomenon suggested that a gas bubble was formed inside the extruder, which was then released at the die plate. It was assumed that this behaviour occurred since the viscosity of the polymer was too low at a temperature setting of 180° C. and thus did not allow for proper mixing with the pressurized carbon dioxide (38). Therefore, it was decided to gradually decrease the temperature settings while gradually increasing the pressure of the injected gas in the extruder. The results of these experiments are shown in Table 6.

TABLE 6
Parameter settings for extrusion experiments of PVP-VA 64
with CO₂ injection - screw configuration 2 and extruder set up 2. Investigation of the effect
of CO₂ pressure and temperature settings on the torque of
the extruder.

| | T₁-T₂ | T₃-T_{die}¹ | P₁ | P₂ | P₃ | Tₘ | N | F | T | Pₚᵤₘₚ |
|---|---|---|---|---|---|---|---|---|---|---|
| Nr. | (° C.) | (° C.) | (bar) | (bar) | (bar) | (° C.) | (rpm) | (kg/h) | (%) | (bar) |
| 1 | 180 | 180 | 3 | 1 | 12 | 186 | 100 | 1 | 31-41 | — |
| 2 | 180 | 170 | 10-22 | 7-20 | 14-22 | 174 | 100 | 1 | 35-45 | 35 |
| 3 | 180 | 160 | 18-28 | 15-25 | 21-31 | 162 | 100 | 1 | 48-59 | 45 |
| 4 | 180 | 150 | 26-34 | 23-31 | 28-36 | 153 | 100 | 1 | 53-69 | 50 |
| 5 | 180 | 140 | 26-42 | 23-40 | 36-56 | 142 | 100 | 1 | 60-80 | 40 |
| 6 | 180 | 130 | 40-49 | 37-45 | 41-49 | 132 | 100 | 1 | 65-80 | 45 |
| 7 | 180 | 120 | 47-55 | 45-53 | 48-56 | 125 | 100 | 1 | 74-92 | 55 |

¹T₃-T_{die}: all zones between T₃ and T_{die} were kept at the same temperature.

When gradually decreasing the temperature, polymer foaming became much more significant and the pressure stabilized during the experiments, i.e. steady state was reached at lower temperature settings. Also, the effect of CO₂ on the torque of the machine was obvious: maximum torque was now reached below 120° C (repeatedly confirmed) compared to 150° C. without gas injection (see Table 5 for comparison). This clearly showed that pressurized CO₂ acted as a plasticizer for PVP-VA 64 and that the processing temperature could be lowered with 30° C. at 1 kg/hr, 100 rpm and injecting carbon dioxide at 55 bar.

To next set of experiments was performed to evaluate the influence of the different parameter settings (feeding rate, screw speed and carbon dioxide pressure) on the torque, polymer foaming, pressure in the extruder and other factors. Table 7 shows these results at 140° C.

TABLE 7
Parameter settings for extrusion experiments of PVP-VA 64
with CO₂ injection - screw configuration 2 and extruder
set up 2. Investigation of the effect of CO₂ pressure,
screw speed and feeding rate on the torque of the extruder.

| | T₁-T₂ | T₃-T_{die}¹ | P₁ | P₂ | P₃ | N | F | T | Pₚᵤₘₚ |
|---|---|---|---|---|---|---|---|---|---|
| Nr. | (° C.) | (° C.) | (bar) | (bar) | (bar) | (rpm) | (kg/h) | (%) | (bar) |
| 1 | 180 | 170 | 2 | 1 | 20 | 100 | 1 | 43-54 | — |
| 2 | 180 | 170 | 9-32 | 7-30 | 11-35 | 100 | 1 | 61-74 | 35 |
| 3 | 180 | 150 | 27-37 | 25-34 | 29-39 | 100 | 1 | 59-71 | 35 |
| 4 | 180 | 140 | 37 | 34 | 32 | 100 | 1 | 72-85 | 35 |
| 5 | 180 | 140 | 41 | 38 | 42 | 100 | 1 | 68-82 | 40 |
| 6 | 180 | 140 | 39-48 | 38-45 | 41-48 | 100 | 1 | 69-88 | 45 |
| 7 | 180 | 140 | 30-54 | 26-52 | 32-55 | 100 | 1 | 73-98 | 50 |
| 8 | 180 | 140 | 34 | 31 | 37 | 100 | 1 | 70-85 | 35 |
| 9 | 180 | 140 | 35 | 33 | 36 | 150 | 1 | 72-85 | 35 |
| 10 | 180 | 140 | 35 | 32 | 36 | 200 | 1 | 71-89 | 35 |
| 11 | 180 | 140 | 35 | 32 | 38 | 100 | 1 | 71-85 | 35 |
| 12 | 180 | 140 | 36 | 33 | 37 | 100 | 0.75 | 70-85 | 35 |
| 13 | 180 | 140 | 35 | 32 | 36 | 100 | 0.5 | 66-80 | 35 |
| 14 | 180 | 140 | 36 | 33 | 37 | 100 | 1 | 71-86 | 35 |
| 15 | 180 | 140 | 35 | 32 | 39 | 100 | 1.25 | 76-92 | 35 |
| 16 | 180 | 140 | 35 | 32 | 39-45 | 100 | 1.5 | 77-96 | 35 |

¹T₃-T_{die}: all zones between T₃ and T_{die} were kept at the same temperature.

Experiments 1-3 were performed to start up the extrusion process and to obtain steady state within the extruder. When doing experiments 4-7 it was observed that maximum foaming and steady state was obtained at 35-40 bar at temperature settings of 140° C., a screw speed of 100 rpm and a feeding rate of 1 kg/hr. A further increase of pressure resulted in lower extent of polymer foaming and periodic pressure drops.

Changing the screw speed (experiments 8-10) resulted in less polymer foaming, but the pressure in the barrel remained constant.

Decreasing the feeding rate (experiments 11-13) also resulted in less polymer foaming, while increasing the feeding rate (experiments 14-16) did not influence the extent of polymer foaming or steady state. At 1.5 kg/hr maximum torque was reached and thus this parameter could not be further explored. Interestingly, the steady state condition and maximal polymer foaming was always obtained in a fast and reproducible way in the different experiments (see experiments 4, 8, 11 and 14). This indicates that steady state was obtained for an optimal set of parameters for temperature, screw speed, feeding rate and carbon dioxide pressure.

Similar experiments were performed at 130° C. and 120° C. to find the condition of steady state (data not shown). At 130° C., steady state was obtained at 40-45 bar with a resulting torque of 80-95% while at 120° C., 60-65 bar resulted in steady state with a torque of 90-100%. Further decreasing the temperature below 120° C., resulted again in a torque above 100% with a subsequent automatic shut down of the machine.

A final set of experiments was performed to evaluate the effect of injecting CO₂ under CFR instead of CPR. Results are shown in Table 8 and 9. At 140° C., steady state was obtained between 0.5 and 8 ml/min. At a flow rate of 15 ml/min, pressure drops started to occur and less polymer foaming was observed. At 30 ml/min, pressure drops were frequently accompanied by gas escaping at the die consistent with bubble formation in the barrel. At 130° C., similar observations were made with pressure drops started already at 8 ml/min. At 10 ml/min they occurred more frequently and at higher CO₂ flow rates, bubble formation became unacceptably high. Interestingly the torque started to decrease at a flow rate of 8 ml/min and higher.

TABLE 8
Parameter settings for extrusion experiments of PVP-VA 64 with CO₂ injection
- screw configuration 2 and extruder set up 2. Investigation of the
effect of CFR on the torque of the machine at 140° C.

| | T₁-T₂ | T₃-T_{die}¹ | P1 | P2 | P3 | N | F | T | CFR |
|---|---|---|---|---|---|---|---|---|---|
| Nr. | (° C.) | (° C.) | (bar) | (bar) | (bar) | (rpm) | (kg/h) | (%) | (ml/min) |
| 1 | 180 | 140 | 34 | 31 | 38 | 100 | 1 | 71-86 | 0.5 |
| 2 | 180 | 140 | 34 | 31 | 38 | 100 | 1 | 69-85 | 1.0 |
| 3 | 180 | 140 | 34 | 31 | 38 | 100 | 1 | 70-85 | 2.0 |
| 4 | 180 | 140 | 34 | 30 | 38 | 100 | 1 | 69-86 | 4.0 |
| 5 | 180 | 140 | 35 | 32 | 38 | 100 | 1 | 68-83 | 8.0 |
| 6 | 180 | 140 | 39-44 | 36-40 | 41-46 | 100 | 1 | 63-83 | 15.0 |
| 7 | 180 | 140 | 44-49 | 42-47 | 43-51 | 100 | 1 | 71-88 | 30.0 |

¹T₃-T_{die}: all zones between T₃ and T_{die} were kept at the same temperature.

TABLE 9
Parameter settings for extrusion experiments of PVP-VA 64 with CO₂ injection
- screw configuration 2 and extruder set up 2. Investigation of the
effect of CFR on the torque of the machine at 130° C.

| | T₁-T₂ | T₃-T_{die1} | P1 | P2 | P3 | N | F | T | CFR |
|---|---|---|---|---|---|---|---|---|---|
| Nr. | (° C.) | (° C.) | (bar) | (bar) | (bar) | (rpm) | (kg/h) | (%) | (ml/min) |
| 1 | 180 | 130 | 36 | 33 | 46 | 100 | 1 | 86-100 | 0.5 |
| 2 | 180 | 130 | 36 | 32 | 49 | 100 | 1 | 87-100 | 1.0 |
| 3 | 180 | 130 | 36 | 32 | 50 | 100 | 1 | 89-100 | 2.0 |
| 4 | 180 | 130 | 42-59 | 39-52 | 52-59 | 100 | 1 | 70-87 | 8.0 |
| 5 | 180 | 130 | 44-65 | 40-62 | 45-66 | 100 | 1 | 70-89 | 10.0 |

¹T₃-T_{die}: all zones between T₃ and T_{die} were kept at the same temperature.

1.3. Physicochemical characteristics of PVP-VA 64 before and after processing with CO₂

The physico-chemical properties of PVP-VA 64 were examined before and after treatment with carbon dioxide, since processing with sub- or supercritical carbon dioxide could have induced changes to the polymer.

Modulated DSC was performed to investigate the thermal characteristics of PVP-VA 64. The glass transition was measured in the reversing signal. The results are shown in Table 10. These results show that there is no difference in glass transition before and after treatment when standard pans are used. When hermetically sealed pans are used, the glass transition is systematically decreased by approximately 25° C. compared with the standard pans. This can be explained by the fact that in standard pans, residual solvent can evaporate. Especially in the case of a Tg above 100° C. and at a heating rate of 2° C./min, all solvent (water) is evaporated by the time the glass transition is reached. On the other hand, when hermetically sealed pans are used, residual solvent can not evaporate. This solvent will act as a plasticizer for the polymer and thus a lower glass transition is obtained. This also explains the difference of approximately 4° C. between samples before and after treatment with carbon dioxide (based on the difference between the average values of the two measurements). These differences are probably due to differences in solvent content rather than differences caused by carbon dioxide treatment.

When the start and end point of the transition are considered (by respectively T₁ and T₂, being the temperatures at which the inflection of the baseline is observed), the range before treatment is comparable to the range after treatment, confirming that no major events happened to the polymer as a function of the treatment.

TABLE 10
Glass transition of PVP-VA 64 before and after treatment with carbon
dioxide. The samples were measured using modulated-DSC and the glass
transition is measured in the reversing signal. Both standard and
hermetically sealed pans were used. Experiments were performed in
duplicate, both values are shown in the table.

| | Before treatment (° C.) | | | After treatment (° C.) | | |
|---|---|---|---|---|---|---|
| | Tg | T₁ | T₂ | Tg | T₁ | T₂ |
| Standard pan | | | | | | |
| 1 | 106.44 | 85.19 | 115.21 | 107.59 | 88.32 | 116.68 |
| 2 | 107.28 | 85.93 | 116.13 | 107.76 | 89.00 | 115.39 |
| AVG | 106.86 | 85.56 | 115.67 | 107.68 | 88.66 | 116.04 |
| Hermetically | | | | | | |
| sealed pan | | | | | | |
| 1 | 79.49 | 72.30 | 91.08 | 72.48 | 65.86 | 79.30 |
| 2 | 70.13 | 64.02 | 77.64 | 68.55 | 64.74 | 76.73 |
| AVG | 74.81 | 68.16 | 84.36 | 70.52 | 65.30 | 78.02 |

TGA analysis was performed to measure any residual solvent loss. Table 11 shows that samples treated with CO₂ contain approximately 1.15% more residual solvent. This explains the difference in glass transition before and after treatment, when measured in hermetically sealed pans.

TABLE 11
Residual solvent loss of PVP-VA 64 before and after
treatment with carbon dioxide. The samples were measured
using TGA. Experiments were performed in duplicate,
both values are shown in the table.

| | Before treatment | After treatment |
|---|---|---|
| | Solvent loss (%) | Solvent loss (%) |
| 1 | 2.29 | 3.49 |
| 2 | 2.34 | 3.46 |

The dissolution of PVP-VA 64 was measured to evaluate whether there was an effect on the dissolution profile before and after treatment with carbon dioxide. Comparison of the dissolution profiles, showed that the samples treated with CO₂ dissolved significantly faster at the 30 minutes time point (t-test, n=3, P<0.05). At all other time points, the dissolution of the carbon dioxide treated polymer was higher, although not significantly. This faster dissolution rate may be attributed to a change in morphology through foam formation when CO₂ is expanded at the exit of the extruder. Particle size of the polymer before and after carbon dioxide treatment was comparable as shown in Table 12. In other words, this parameter could not be the source of the differences observed during dissolution measurement.

TABLE 12
Particle size of PVP-VA 64 before and after treatment
with carbon dioxide. The particle size was measured
using the vibrating sieve method.

| | Before treatment | After treatment |
|---|---|---|
| d84 | 104μ | 80μ |
| d50 | 158μ | 156μ |
| <100μ | 38.3% | 42.7% |

Measurement of the specific surface area showed that the processed sample had a specific surface area of 0.381 m²/g compared to 0.261 m²/g for the unprocessed sample. Since particle size was comparable, there had to be another parameter that caused this difference in specific surface area. To investigate this further in detail, light microscopy and SEM were performed. The light photomicrograph showed that the foam consisted of thin walls connected to each other.

The SEM figures clearly showed that the morphology of the carbon dioxide treated polymer had changed from sphere like particles, before treatment, to very thin platelets after treatment. These platelets were formed during the foaming step when CO₂ expanded at the exit of the extruder. Although the vibrating sieve method indicated that there was no difference in particle size, the particle size of the treated polymer was probably overestimated, due to the shape of the platelets (the platelets may be retained by the sieves depending on their position). This also explained the difference in specific surface area and dissolution.

1.4. Melt extrusion of itraconazole/PVP-VA 64 10/90

1.4.1. Melt extrusion of itraconazole/P VP-VA 64 10/90 without CO₂ injection

The parameter settings and resulting torques are listed in Table 13. Maximum torque was reached below 140° C. when the temperature of all zones is decreased gradually while keeping screw speed (100 rpm) and feeding rate (1 kg/hr) constant (experiments 1-4 in Table 13). When the first two zones were maintained at increased temperature (180° C.), maximum torque was achieved below 120° C. with a feeding rate of 1 kg/hr and a screw speed of 100 rpm (experiments 4-9 in Table 13).

TABLE 13
Parameter settings for extrusion experiments of Itraconazole/PVP-VA
64 10/90 without CO₂ injection. Investigation of the effect of
the temperature settings on the torque of the extruder.

| | T₁-T₂ | T₃-T_{die}¹ | P₁ | P₂ | P₃ | Tₘ | n | F | |
|---|---|---|---|---|---|---|---|---|---|
| Nr. | (° C.) | (° C.) | (bar) | (bar) | (bar) | (° C.) | (rpm) | (kg/h) | T (%) |
| 1 | 180 | 180 | 3 | 3 | 8 | 190 | 100 | 1.0 | 26-34 |
| 2 | 160 | 160 | 2 | 2 | 13 | 162 | 100 | 1.0 | 38-49 |
| 3 | 140 | 140 | 1 | 2 | 32 | 144 | 100 | 1.0 | 84-98 |
| 4 | 135 | 135 | — | — | — | — | 100 | 1.0 | >100 |
| 6 | 180 | 160 | 2 | 2 | 11 | 165 | 100 | 1.0 | 30-38 |
| 7 | 180 | 140 | 2 | 2 | 37 | 146 | 100 | 1.0 | 50-62 |
| 8 | 180 | 125 | 2 | 1 | 76 | 137 | 100 | 1.0 | 86-100 |
| 9 | 180 | 120 | — | — | — | — | 100 | 1.0 | >100 |

¹T₃-T_{die}: all zones between T₃ and T_{die} were kept at the same temperature.

1.4.2. Melt extrusion of itraconazole/PVP-VA 64 10/90 with CO₂ injection

The first set of experiments with the injection of carbon dioxide while extruding itraconazole/PVP-VA 64 10/90 was performed to find the minimal working temperature while gradually decreasing the temperature settings. The results of the experiments are shown in Table 14.

TABLE 14
Parameter settings for extrusion experiments of Itraconazole/PVP-VA
64 10/90 with CO₂ injection. Investigation of the effect of CO₂ pressure
and temperature settings on the torque of the extruder.

| | T₁-T₂ | T₃-T_{die}¹ | P₁ | P₂ | P₃ | Tₘ | N | F | | Pₚᵤₘₚ |
|---|---|---|---|---|---|---|---|---|---|---|
| Nr. | (° C.) | (° C.) | (bar) | (bar) | (bar) | (° C.) | (rpm) | (kg/h) | T (%) | (bar) |
| 1 | 160 | 160 | 18 | 16 | 20 | 161 | 100 | 1.0 | 50-63 | 35 |
| 2 | 140 | 140 | 30 | 28 | 30 | 142 | 100 | 1.0 | 62-75 | 35 |
| 3 | 135 | 135 | 20 | 22 | 25 | 139 | 100 | 1.0 | 74-88 | 35 |
| 4 | 130 | 130 | — | — | — | — | 100 | 1.0 | >100 | 35 |
| 5 | 180 | 140 | 30 | 28 | 30 | 142 | 100 | 1.0 | 55-69 | 35 |
| 6 | 180 | 130 | 35 | 33 | 35 | 132 | 100 | 1.0 | 59-75 | 35 |
| 7 | 180 | 120 | 35 | 33 | 37 | 124 | 100 | 1.0 | 72-86 | 37 |
| 8 | 180 | 115 | 35 | 33 | 54 | 121 | 100 | 1.0 | 81-94 | 38 |
| 9 | 180 | 110 | 35 | 33 | 53 | 118 | 100 | 1.0 | >100 | 38 |

¹T₃-T_{die}: all zones between T₃ and T_{die} were kept at the same temperature.

Under these conditions, polymer foaming was observed and the pressure stabilized during the experiments, i.e. steady state was reached. The processing temperature could be lowered by 10° C. (repeatedly confirmed). This showed that pressurized CO₂ acted also as a plasticizer for itraconazole/PVP-VA 64 10/90 and that the processing temperature could be lowered up to 10° C. at 1 kg/hr, 100 rpm and injecting carbon dioxide at 35-40 bar, i.e. under subcritical conditions. Compared to pure PVP-VA 64 the processing temperature could be lowered up to 30° C. at 1 kg/hr, 100 rpm and injecting carbon dioxide at 35-40 bar. This reduced plasticising effect of carbon dioxide is probably due to the presence of itraconazole, which already partly plasticises the polymer leaving less influence for carbon dioxide.

1.4.3. Physicochemical characteristics of itraconazole/PVP-VA 64 10/90 before and after processing with CO₂

The physico-chemical properties were examined before processing and after extrusion with and without injection of carbon dioxide, since processing with sub- or supercritical carbon dioxide could have induced changes to the polymer.

The following samples were selected for evaluation:

Samples 1, 2, 3 and 8 of Table 13 and samples 1, 2, 3, 5, 7 and 9 from Table 14.

Results of the modulated-DSC experiments are shown in Table 15 and 16. The hermetically sealed pans resulted in a lot of noise in the DSC profile, making interpretation very difficult. Therefore, only the results of the standard pans are shown in the tables.

These results show that there is no change in the glass transition and the heat capacity between the samples processed with and without carbon dioxide injection. All DSC profiles lack the melting enthalpy of itraconazole indicating the formation of an amorphous dispersion. Calculation of the theoretical Tg according to the Fox equation results in a value of 376 K (T_{g,itraconazole}=332 K and T_{g,PVP-VA 64}=382 K), which is equal to the experimental Tg values. This indicates that itraconazole and PVP-VA 64 in a drug/carrier ratio of 10/90 w/w are completely miscible without phase separation. This seems not to be influenced by injecting carbon dioxide during the extrusion process.

TABLE 15
Glass transition of itraconazole/PVP-VA 64 10/90 measured using
modulated-DSC and the glass transition is measured in the reversing
signal. Results of the standard pans are presented in the table.
Experiments were performed in duplicate.

| | Before CO₂ injection | | | |
|---|---|---|---|---|
| Standard pan | Sample 13-2 | Sample 13-3 | Sample 13-8 | — |
| 1 | 104.14 | 105.39 | 103.27 | — |
| 2 | 104.47 | 103.52 | 103.23 | — |
| AVG | 104.31 | 104.46 | 103.25 | — |

| | After CO₂ injection | | | |
|---|---|---|---|---|
| Standard pan | Sample 14-1 | Sample 14-3 | Sample 14-7 | Sample 14-9 |
| 1 | 103.19 | 104.68 | 103.97 | 102.72 |
| 2 | 104.59 | 104.96 | 104.66 | 103.54 |
| AVG | 104.25 | 104.82 | 104.32 | 103.13 |

TABLE 16
Heat capacity (J/g. ° C.) of the glass transition of itraconazole/PVP-VA
64 10/90 measured using modulated-DSC in the reversing signal.
Results of the standard pans are presented in the table. Experiments
were performed in duplicate.

| | Before CO₂ injection | | | |
|---|---|---|---|---|
| Standard pan | Sample 13-2 | Sample 13-3 | Sample 13-8 | — |
| 1 | 0.374 | 0.327 | 0.377 | — |
| 2 | 0.377 | 0.342 | 0.335 | — |
| AVG | 0.376 | 0.335 | 0.356 | — |

| | After CO₂ injection | | | |
|---|---|---|---|---|
| Standard pan | Sample 14-1 | Sample 14-3 | Sample 14-7 | Sample 14-9 |
| 1 | 0.380 | 0.400 | 0.371 | 0.352 |
| 2 | 0.383 | 0.405 | 0.404 | 0.380 |
| AVG | 0.382 | 0.403 | 0.388 | 0.366 |

TGA analysis was performed to measure any residual solvent loss. Table 5 shows that there are only minor differences in residual solvent for the samples processed with and without carbon dioxide injection.

TABLE 17
Residual solvent loss of itraconazole/PVP-VA 64 10/90 before
and after treatment with carbon dioxide. The samples were measured
using TGA. Experiments were performed in duplicate

Before CO₂ injection

| | Sample 13-2 | Sample 13-3 | Sample 13-8 | — |
|---|---|---|---|---|
| 1 | 2.64 | 3.59 | 3.96 | — |
| 2 | 2.65 | 3.69 | 4.14 | — |
| AVG | 2.65 | 3.64 | 4.05 | — |

| | After CO₂ injection | | | |
|---|---|---|---|---|
| | Sample 14-1 | Sample 14-3 | Sample 14-7 | Sample 14-9 |
| 1 | 3.33 | 3.82 | 5.32 | 4.42 |
| 2 | 3.01 | 4.16 | 5.28 | 4.58 |
| AVG | 3.17 | 3.99 | 5.30 | 4.50 |

Light microscopy was performed to study the macroscopic morphology of the samples. The samples extruded at 140° C. without carbon dioxide injection contained crystalline itraconazole, while the sample extruded at 180° C. was completely clear. Since itraconazole melts at about 165° C., this may indicate that the spots represent crystalline itraconazole in the polymer matrix. Microscopy shows that the foam characteristics are changed as a function of temperature settings and pressure of the injected carbon dioxide. These samples do not show the presence of crystalline itraconazole. However, interpretation is difficult due to the foam morphology of the samples. None of the DSC profiles indicated presence of crystalline itraconazole. This may be due to the fact that itraconazole dissolves in the polymer matrix while heating the sample in the DSC oven or that the concentration of the crystalline itraconazole is too low to detect. Milling of these different products resulted in different morphologies for the milled samples as well. The milled foam consisted of thinner flakes compared to the milled extrudate strands produced without carbon dioxide injection. This resulted in a different bulk and tapped density. The bulk and tapped density for the milled extrudate before carbon dioxide injection were 0.482 g/ml and 0.576 g/ml, respectively (compressibility 16.3%). While for the carbon dioxide treated extrudate, these values became 0.130 g/ml and 0.206 ml/g, respectively (compressibility 36.9%). This means that compressibility is improved after carbon dioxide injection, but that powder flow has decreased.

The adsorption/desorption profile was recorded for samples 13-3, 13-8, 13-3 and 14-7. Samples 13-3 and 13-8 adsorbed lesser water compared to carbon dioxide treated material, i.e. samples 14-3 and 14-7 (˜28% versus ˜46%). Probably, this also can be explained by the different morphology.

The dissolution of the different samples was first measured with a 200 mg dose, that is 2 g of extrudate. This amount of material in combination with poor wettability, resulted in a lot of variability and irreproducible analytical results. Therefore a 50 mg dose was measured. The mean values for the release after 60 minutes are given in Table 18. The table shows that there is a significant difference between the physical mixture, the samples before carbon dioxide injection and the samples after carbon dioxide injection. The carbon dioxide treated material shows a slower dissolution compared to the untreated material, i.e. the release of itraconazole was controlled. Dissolution seemed not to be influenced by the temperature settings.

TABLE 18
Dissolution results after 60 minutes for the different samples. The
mean values and the standard deviation (STDEV) are given. Different
superscripts denote significantly different means calculated using
ANOVA and a post hoc multiple range test (p < 0.05).

| Sample | Release after 60′ (AVG ± STDEV) |
|---|---|
| Physical mixture | 8.4 ± 0 |
| 13-2 | 88.5 ± 5.8 |
| 13-3 | 92.9 ± 1.8 |
| 13-8 | 91.6 ± 6.7 |
| 14-2 | 68.9 ± 22.0 |
| 14-3 | 69.6 ± 3.4 |
| 14-7 | 71.0 ± 10.7 |
| 14-9 | 68.5 ± 10.5 |

1.5. Melt extrusion of itraconazole/PVP-VA 64 40/60

1.5.1. Melt extrusion of itraconazole/PVP-VA 64 40/60 without CO₂ injection

The parameter settings and resulting torques are listed in Table 13. Maximum torque was reached below 135° C. when the temperature of all zones was decreased gradually while keeping screw speed (100 rpm) and feeding rate (1 kg/hr) constant (experiments 1-5 in Table 19). When the first two zones were maintained at increased temperature (180° C.), maximum torque was achieved below 110° C. with a feeding rate of 1 kg/hr and a screw speed of 100 rpm (experiments 6-10 in Table 19).

TABLE 19
Parameter settings for extrusion experiments of Itraconazole/PVP-VA
64 40/60 without CO₂ injection. Investigation of the effect of
the temperature settings on the torque of the extruder.

| | T₁-T₂ | T₃-T_{die}¹ | P₁ | P₂ | P₃ | Tₘ | n | F | T |
|---|---|---|---|---|---|---|---|---|---|
| Nr. | (° C.) | (° C.) | (bar) | (bar) | (bar) | (° C.) | (rpm) | (kg/h) | (%) |
| 1 | 180 | 180 | 3 | 2 | 6 | 188 | 100 | 1.0 | 23-29 |
| 2 | 160 | 160 | 2 | 1 | 8 | 166 | 100 | 1.0 | 29-38 |
| 3 | 140 | 140 | 1 | 1 | 28 | 145 | 100 | 1.0 | 69-83 |
| 4 | 135 | 135 | 1 | 1 | 32 | 141 | 100 | 1.0 | 80-98 |
| 5 | 130 | 130 | — | — | — | — | 100 | 1.0 | >100 |
| 6 | 180 | 160 | 2 | 2 | 7 | 164 | 100 | 1.0 | 25-33 |
| 7 | 180 | 140 | 2 | 1 | 15 | 142 | 100 | 1.0 | 33-44 |
| 8 | 180 | 120 | 1 | 1 | 49 | 127 | 100 | 1.0 | 62-74 |
| 9 | 180 | 110 | 1 | 1 | 67 | 123 | 100 | 1.0 | 81-97 |
| 10 | 180 | 105 | — | — | — | — | 100 | 1.0 | >100 |

¹T₃-T_{die}: all zones between T₃ and T_{die} were kept at the same temperature.

1.5.2. Melt extrusion of itraconazole/PVP-VA 64 40/60 with CO₂ injection

The experiments with the injection of carbon dioxide while extruding itraconazole/PVP-VA 64 40/60 were performed to find the minimal working temperature while gradually decreasing the temperature settings. The results of the experiments are shown in Table 20.

TABLE 20
Parameter settings for extrusion experiments of Itraconazole/PVP-VA
64 40/60 with CO₂ injection. Investigation of the effect of CO₂ pressure
and temperature settings on the torque of the extruder.

| | T₁-T₂ | T₃-T_{die}¹ | P₁ | P₂ | P₃ | Tₘ | N | F | T | Pₚᵤₘₚ |
|---|---|---|---|---|---|---|---|---|---|---|
| Nr. | (° C.) | (° C.) | (bar) | (bar) | (bar) | (° C.) | (rpm) | (kg/h) | (%) | (bar) |
| 1 | 160 | 160 | 4-6 | 3-5 | 4-7 | 165 | 100 | 1.0 | 27-38 | 40 |
| 2 | 140 | 140 | 17 | 15 | 17 | 142 | 100 | 1.0 | 55-68 | 40 |
| 3 | 130 | 130 | 24 | 23 | 24 | 134 | 100 | 1.0 | 66-81 | 40 |
| 4 | 125 | 125 | — | — | — | — | 100 | 1.0 | >100 | 40 |
| 5 | 180 | 140 | 2-9 | 2-8 | 9-11 | 146 | 100 | 1.0 | 32-42 | 40 |
| 6 | 180 | 120 | 16-28 | 25-26 | 24-25 | 122 | 100 | 1.0 | 49-60 | 38 |
| 7 | 180 | 110 | 32 | 30 | 63 | 117 | 100 | 1.0 | 65-79 | 38 |
| 8 | 180 | 105 | 32 | 30 | 79 | 115 | 100 | 1.0 | 78-95 | 39 |
| 9 | 180 | 100 | — | — | — | — | 100 | 1.0 | >100 | 39 |

¹T₃-T_{die}: all zones between T₃ and T_{die} were kept at the same temperature.

Under these conditions, polymer foaming was observed and the pressure stabilized during the experiments, i.e. steady state was reached. The processing temperature could be lowered by 5° C. (repeatedly confirmed). This showed that pressurized CO₂ acted also as a plasticizer for itraconazole/PVP-VA 64 40/60 and that the processing temperature could be lowered up to 5° C. at 1 kg/hr, 100 rpm and injecting carbon dioxide at 35-40 bar, i.e. under subcritical conditions. Compared to pure PVP-VA 64 the processing temperature could be lowered up to 30° C. at 1 kg/hr, 100 rpm and injecting carbon dioxide at 35-40 bar. This reduced plasticising effect of carbon dioxide is probably due to the presence of itraconazole, which already partly plasticises the polymer leaving less influence for carbon dioxide.

1.5.3. Physicochemical characteristics of itraconazole/PVP-VA 64 40/60 before and after processing with CO₂

The physico-chemical properties were examined before processing and after extrusion with and without injection of carbon dioxide, since processing with sub- or supercritical carbon dioxide could have induced changes to the polymer. The following samples were selected for evaluation: Samples 3 and 8 of Table 19 and samples 2 and 7 from Table 20.

Results of the modulate DSC experiments are shown in Tables 21 and 22. These results show that there is a difference in the glass transition as a function of processing temperature. When extruded below the melting point of itraconazole, the Tg was higher compared to samples prepared above the melting point. Calculation of the theoretical Tg according to the Fox equation results in a value of 360 K (T_{g,itraconazole}=332 K and T_{g,PVP-VA 64}=382 K), which is slightly lower than the experimental Tg values. The DSC profiles of the samples prepared below the melting point of the drug substance, show the existence of a melting endotherm of itraconazole in the samples at room temperature. Based on the melting enthalpy of pure itraconazole (ΔH=85 J/g), the percentage crystalline itraconazole in the samples can be calculated (see Table 22).

Power XRD confirmed the presence of crystalline itraconazole in those samples that were prepared below the melting point of itraconazole.

These results indicate that itraconazole and PVP-VA 64 in a drug/carrier ratio of 40/6-w/w are completely miscible when prepared at temperature settings above the melting point of itraconazole, but phase separation occurs when processed below the melting point. Injection of carbon dioxide during the extrusion process does not seem to influence the thermal characteristics of the samples.

TABLE 21
Glass transition of itraconazole/PVP-VA 64 40/60 measured using
modulated-DSC and the glass transition is measured in the reversing
signal. Results of the standard pans are presented in the table.
Experiments were performed in duplicate.
Before CO₂ injection

| Standard pan | Sample 19-3 | Sample 19-8 |
|---|---|---|
| 1 | 94.4 | 90.2 |
| 2 | 93.5 | 89.4 |
| AVG | 94.0 | 89.8 |

After CO₂ injection

| Standard pan | Sample 20-2 | Sample 20-7 |
|---|---|---|
| 1 | 93.8 | 90.0 |
| 2 | 93.7 | 88.7 |
| AVG | 93.8 | 89.4 |

TABLE 22
Heat capacity (J/g. ° C.) of the glass transition of itraconazole/
PVP-VA 64 40/60 measured using modulated-DSC in the reversing
signal. Results of the standard pans are presented in the table.
Experiments were performed in duplicate.
Before CO₂ injection

| | |
|---|---|
| Sample 19-3 | Sample 19-8 |

| | ΔH (J/g) | % cryst. | Δ(J/g) |
|---|---|---|---|
| 1 | 11.8 | 34.7 | n.d. |
| 2 | 11.7 | 34.4 | n.d. |
| AVG | 11.8 | 34.6 | n.d. |

After CO₂ injection

| | |
|---|---|
| Sample 20-2 | Sample 20-7 |

| Standard pan | ΔH (J/g) | % cryst. | Δ(J/g) |
|---|---|---|---|
| 1 | 12.1 | 35.6 | n.d. |
| 2 | 11.3 | 33.2 | n.d. |
| AVG | 11.7 | 34.4 | n.d. |

n.d. = not detectable

TGA analysis was performed to measure any residual solvent loss. Table 23 shows that there are no differences in residual solvent for the samples processed with and without carbon dioxide injection.

TABLE 23
Residual solvent loss of itraconazole/PVP-VA 64 40/60 before
and after treatment with carbon dioxide. The samples were measured
using TGA. Experiments were performed in duplicate
Before CO₂ injection

| | Sample 19-3 | Sample 19-8 |
|---|---|---|
| 1 | 1.68 | 2.12 |
| 2 | 1.62 | 1.98 |
| AVG | 1.65 | 2.05 |

After CO₂ injection

| | Sample 20-2 | Sample 20-7 |
|---|---|---|
| 1 | 1.68 | 1.63 |
| 2 | 1.43 | 1.59 |
| AVG | 1.56 | 1.61 |

Light microscopy was performed to study the macroscopic morphology of the samples. The sample extruded at 140° C. without carbon dioxide injection was completely white, while the sample extruded above the melting point of itraconazole was completely clear. This confirms the existence of phase separation in the samples extruded below the melting point of itraconazole. The foam characteristics could have been changed as a function of temperature settings and pressure of the injected carbon dioxide. Polarized light microscopy, clearly showed birefrigency for the samples prepared below the melting point of itraconazole, also confirming phase separation. Milling of these different products resulted in different morphologies for the milled samples as well. The milled foam consisted of thinner flakes compared to the milled extrudate strands produced without carbon dioxide injection. This resulted in a different bulk and tapped density. The bulk and tapped density for the milled extrudate before carbon dioxide injection were 0.500 g/ml and 0.625 g/ml, respectively (compressibility 20.0%). While for the carbon dioxide treated extrudate, these values became 0.351 g/ml and 0.492 ml/g, respectively (compressibility 28.7%). This means that compressibility improved after carbon dioxide injection, but that powder flow decreased.

The mean values for the release after 60 minutes are given in Table 24. The table shows that there is a significant difference between the physical mixture and all extrudate samples. These data suggest there is a significant influence of the temperature settings and the injection of carbon dioxide on the dissolution properties of extrudates. Samples prepared above the melting point of itraconazole, result in a faster release as well as samples not treated with carbon dioxide. This means that release of itraconazole can be controlled by the processing conditions of the extrusion process.

TABLE 24
Dissolution results after 60 minutes for the different samples. The
mean values and the standard deviation (STDEV) are given. Different
superscripts denote significantly different means calculated using
ANOVA and a post hoc multiple range test (p < 0.05).

| Sample | Release after 60′ (AVG ± STDEV) |
|---|---|
| Physical mixture | 2.4 ± 0.05 |
| 19-2 | 30.7 ± 8.2 |
| 19-8 | 72.1 ± 4.2 |
| 20-2 | 15.9 ± 1.3 |
| 20-7 | 45.4 ± 1.3 |

Table 25 gives a comparison of the minimal temperature settings during the extrusion process as a function of the Tg for pure PVP-VA 64, itraconazole/PVP-VA 64 10/90 and 40/60. These results show that both itraconazole as well as carbon dioxide act as a plasticizer for PVP-VA 64. The total effect of both components is for all three systems comparable, i.e. the minimal temperature settings are between 11° C. and 13° C. above the glass transition of the samples.

TABLE 25
Comparison of the minimal temperature settings versus Tg for
pure PVP-PA 64, itraconazole/PVP-VA 64 10/90 and 40/60.

| | | itraconazole/ | itraconazole/ |
|---|---|---|---|
| | PVP-VA | PVP-VA 64 | PVP-VA 64 |
| | 64 | 10/90 | 40/60 |
| Tg (° C.)¹ | 109 | 104 | 92 |
| T_{sct, min} (° C.)² | 150 | 125 | 110 |
| T_{sct, min, CO2} (° C.)³ | 120 | 115 | 105 |
| T_{sct, min, CO2} -Tg (° C.) | 11 | 11 | 13 |

¹Tg = means glass transition of the sample
²T_{sct, min} = minimal temperature settings of the barrel during melt extrusion without injection of carbon dioxide (T_{1, 2} = 180° C., F = 1 kg/hr, n = 100 rpm)
³T_{set, min, CO2} = minimal temperature settings of the barrel during melt extrusion and injection of carbon dioxide (T_{1.2} = 180° C., F = 1 kg/hr, n = 100 rpm)

1.6. Conclusion for PVP-VA 64

Based on the experiments with PVP-VA 64, an extruder set up and screw configuration were found which allowed for the injection of pressurized carbon dioxide. Injection of the pressurized gas, building up pressure and polymer foaming were obtained with an extruder and screw set up whereby a melt seal was obtained using a reversing transport element and the die opening and whereby CO₂ was expanded after the die plate. A method was also established to obtain steady state and significant polymer foaming upon release of the pressure. This method consists of gradually decreasing the temperature in the barrel while increasing the pressure of the injected gas. Furthermore, it can be concluded that CO₂ acts as a plasticizer for PVP-VA 64 since the process temperature can be lowered with at least 30° C. Steady state is obtained as a function of optimal pump pressure and temperature settings. The maximal pressure that could be obtained with PVP-VA 64 was approximately 65 bar, which means that the extrusion was performed under subcritical conditions.

The physicochemical characterization of the polymer revealed that the specific surface area was increased due to a morphology change, which probably provided for increased dissolution of the polymer. Other characteristics such as the glass transition did not change as a function of carbon dioxide treatment.

The extruder set up and screw configuration that were found to be optimal for the polymers PCP-VA 64, Eudragit E100 PO, also worked for itraconazole/PVP-VA 64 10/90 w/w and 40/60 w/w. A method to obtain steady state conditions and significant polymer foaming upon release of the pressure could be identified. This method consists of gradually decreasing the temperature in-the barrel while increasing the pressure of the injected gas.

Furthermore, it can be concluded the CO₂ acts as a plasticizer for itraconazole/PVP-VA 64 10/90 w/w since the process temperature could be lowered by at least 10° C., as well as for itraconazole/PVP-VA 64 40/60 w/w since the process temperature could be lowered by at least 5° C. Steady state was obtained as a function of optimal pump pressure and temperature settings. The maximal pressure that could be obtained was approximately 40 bar, which means that the extrusion in this case was performed under subcritical conditions.

The physicochemical characterization of the extrudates revealed that the foam morphology was changed as a function of CO₂ treatments. This resulted in different particle morphology (platelet shape) and bulk and tapped density (improved compressibility, >25%). DSC measurements indicated the formation of a completely miscible amorphous system (following the Fox equation), i.e. the formation of an amorphous solution. Also after injection of the carbon dioxide, the system was still amorphous. However, extrudates that were produced below the melting temperature of itraconazole, showed presence of crystalline itraconazole. Injecting carbon dioxide did not seem to influence the thermal properties of the samples.

The dissolution of the extrudates was increased compared to the physical mixture and could be controlled by treatment with carbon dioxide.

2. Eudragit E100 PO

From the experiments performed with PVP-VA 64, a screw configuration and an extruder set up were found that allowed for (a) the injection of pressurized carbon dioxide, (b) the build up of pressure inside the extruder and, (c) the creation of a foam upon expansion of the carbon dioxide at the exit of the extruder. This screw configuration and extruder set up, as shown in FIGS. 2 and 4, were used for extrusion trials with Eudragit E100 PO as well. The experiments were performed with and without carbon dioxide injection. The trials with CO₂ injection were completed and finally the physicochemical characteristics of Eudragit E100 PO before and after treatment were investigated.

2.1. Melt extrusion of Eudragit E100 PO without CO₂ injection

The parameter settings and resulting torques are listed in Table 26. Maximum torque was reached at 140° C. when the temperature of all zones was decreased gradually while keeping screw speed (150 rpm) and feeding rate (1 kg/hr) constant (experiments 11 -14 in). When the first two zones were maintained at increased temperature (180° C.), maximum torque was achieved at 130° C. with a feeding rate of 1 kg/hr and a screw speed of 100 rpm (experiments 15-20 in Table 26). Furthermore it was observed that an increase of the feeding rate caused an increase in the torque (experiments 6-10 in Table 26), which is as expected and is consistent with results obtained from PVP-VA 64. However, at 2.5 kg/hr, bridging occurred in the inlet funnel which caused blockage of the powder feed. This phenomenon occurs with poorly flowing powders. Therefore the maximum allowable feeding rate with Eudragit E100 PO was 2 kg/hr (with this type of extruder).

An increase of the screw speed resulted in a decreased torque (experiments 1-5 in Table 26), which was as expected, and contrary to the observations made during the trials with PVP-VA 64.

In the case of Eudragit E100 PO, the melt viscosity was measured as a function of shear rate (frequency in rad/s) and temperature. These profiles clearly showed that the apparent viscosity decreased with increasing shear rate and thus, it could be concluded that Eudragit E100 PO showed shear thinning behaviour.

In addition, it was clear that the viscosity increased with decreasing temperature, which was consistent with the observations made during the extrusion trials.

TABLE 26
Parameter settings for extrusion experiments of Eudragit E100 PO without CO₂ injection - screw configuration 2 and extruder
set up 2. Investigation of the effect of screw speed, feeding rate and temperature settings on the torque of the extruder.

| | T₁ | T₂ | T₃ | T₄ | T₅ | T₆ | T₇ | T_{flange} | T_{die} | P₁ | P₂ | P₃ | Tₘ | n | F | T |
|---|---|---|---|---|---|---|---|---|---|---|---|---|---|---|---|---|
| Nr. | (° C.) | (° C.) | (° C.) | (° C.) | (° C.) | (° C.) | (° C.) | (° C.) | (° C.) | (bar) | (bar) | (bar) | (° C.) | (rpm) | (kg/hr) | (%) |
| 1 | 150 | 160 | 160 | 170 | 170 | 170 | 170 | 170 | 170 | 2 | 1 | 8 | 177 | 50 | 0.5 | 67-89 |
| 2 | 150 | 160 | 160 | 170 | 170 | 170 | 170 | 170 | 170 | 2 | 1 | 8 | 178 | 100 | 0.5 | 54-70 |
| 3 | 150 | 160 | 160 | 170 | 170 | 170 | 170 | 170 | 170 | 2 | 1 | 8 | 178 | 150 | 0.5 | 47-64 |
| 4 | 150 | 160 | 160 | 170 | 170 | 170 | 170 | 170 | 170 | 2 | 1 | 9 | 178 | 200 | 0.5 | 44-60 |
| 5 | 150 | 160 | 160 | 170 | 170 | 170 | 170 | 170 | 170 | 2 | 1 | 9 | 178 | 250 | 0.5 | 41-57 |
| 6 | 160 | 160 | 160 | 170 | 170 | 170 | 170 | 170 | 170 | 2 | 1 | 8 | 176 | 150 | 0.5 | 42-58 |
| 7 | 160 | 160 | 160 | 170 | 170 | 170 | 170 | 170 | 170 | 2 | 1 | 11 | 176 | 150 | 1.0 | 50-68 |
| 8 | 160 | 160 | 160 | 170 | 170 | 170 | 170 | 170 | 170 | 2 | 1 | 14 | 176 | 150 | 1.5 | 55-73 |
| 9 | 160 | 160 | 160 | 170 | 170 | 170 | 170 | 170 | 170 | 2 | 1 | 16 | 176 | 150 | 2.0 | 58-78 |
| 10 | 160 | 160 | 160 | 170 | 170 | 170 | 170 | 170 | 170 | — | — | — | 176 | 150 | 2.5 | — |
| 11 | 170 | 170 | 170 | 170 | 170 | 170 | 170 | 170 | 170 | 2 | 1 | 12 | 177 | 150 | 1.0 | 50-66 |
| 12 | 160 | 160 | 160 | 160 | 160 | 160 | 160 | 160 | 160 | 2 | 1 | 16 | 166 | 150 | 1.0 | 57-76 |
| 13 | 150 | 150 | 150 | 150 | 150 | 150 | 150 | 150 | 150 | 2 | 1 | 19 | 156 | 150 | 1.0 | 68-88 |
| 14 | 140 | 140 | 140 | 140 | 140 | 140 | 140 | 140 | 140 | 2 | 0 | 27 | 148 | 150 | 1.0 | 85-100 |
| 15 | 180 | 180 | 180 | 180 | 180 | 180 | 180 | 180 | 180 | 2 | 1 | 9 | 187 | 100 | 1.0 | 49-71 |
| 16 | 180 | 180 | 170 | 170 | 170 | 170 | 170 | 170 | 170 | 2 | 1 | 12 | 175 | 100 | 1.0 | 54-73 |
| 17 | 180 | 180 | 160 | 160 | 160 | 160 | 160 | 160 | 160 | 2 | 1 | 15 | 165 | 100 | 1.0 | 57-76 |
| 18 | 180 | 180 | 150 | 150 | 150 | 150 | 150 | 150 | 150 | 2 | 1 | 20 | 156 | 100 | 1.0 | 62-82 |
| 19 | 180 | 180 | 140 | 140 | 140 | 140 | 140 | 140 | 140 | 2 | 0 | 30 | 145 | 100 | 1.0 | 73-91 |
| 20 | 180 | 180 | 130 | 130 | 130 | 130 | 130 | 130 | 130 | 1 | 0 | 41 | 136 | 100 | 1.0 | 86-100 |

2.2. Melt extrusion of Eudragit E100 PO with CO₂ injection

The first set of experiments with the injection of carbon dioxide while extruding Eudragit E100 PO was performed to find the minimal working temperature while gradually decreasing the temperature settings. When doing this, the first two zones were kept constant (180° C.) as was the feeding rate (1 kg/hr) and screw speed (100 rpm). The results of the experiments are shown in Table 27.

TABLE 27
Parameter settings for extrusion experiments of Eudragit E100
PO with CO₂ injection - screw configuration 2 and extruder set up 2. Investigation of the
effect of CO₂ pressure and temperature settings on the torque
of the extruder.

| | T₁-T₂ | T₃-T_{die}¹ | P₁ | P₂ | P₃ | Tₘ | N | F | T | Pₚᵤₘₚ |
|---|---|---|---|---|---|---|---|---|---|---|
| Nr. | (° C.) | (° C.) | (bar) | (bar) | (bar) | (° C.) | (rpm) | (kg/h) | (%) | (bar) |
| 1 | 180 | 170 | 9-14 | 3-7 | 8-13 | 175 | 100 | 1 | 51-71 | 30 |
| 2 | 180 | 160 | 11-14 | 8-12 | 13-16 | 164 | 100 | 1 | 55-74 | 30 |
| 3 | 180 | 150 | 15-16 | 10-12 | 16-18 | 153 | 100 | 1 | 58-80 | 20 |
| 4 | 180 | 140 | 22-26 | 19-22 | 22-26 | 144 | 100 | 1 | 64-83 | 35 |
| 5 | 180 | 130 | 27-32 | 24-28 | 26-31 | 134 | 100 | 1 | 69-89 | 38 |
| 6 | 180 | 120 | 34 | 31 | 34 | 122 | 100 | 1 | 77-98 | 38 |
| 7 | 180 | 115 | 44 | 41 | 44 | 118 | 100 | 1 | 82-99 | 45 |
| 8 | 180 | 110 | — | — | — | 115 | 100 | 1 | >100 | 40 |

¹T₃-T_{die}: all zones between T₃ and T_{die} were kept at the same temperature.

Under these conditions, it was observed that polymer foaming became much more significant and the pressure stabilized during the experiments, i.e. steady state was reached. Also, the effect of CO₂ on the torque of the machine was obvious: maximum torque was now reached below 115° C. (repeatedly confirmed) compared to 130° C. without gas injection (see Table 26 for comparison). This showed that pressurized CO₂ acted also as a plasticizer for Eudragit E100 PO and that the processing temperature could be lowered by 15° C. at 1 kg/hr, 100 rpm and injecting carbon dioxide at 40-45 bar.

Compared to PVP-VA 64, polymer foaming was less pronounced with Eudragit E100 PO and temperature could be lowered by 15° C., instead of the 30° C. with PVP-VA 64.

The next set of experiments was performed to evaluate the influence of the different parameter settings (feeding rate, screw speed and carbon dioxide pressure) on the torque, polymer foaming, pressure in the extruder, and other factors. Table 28 shows these results at 120° C.

TABLE 28
Parameter settings for extrusion experiments of
Eudragit E100 PO with CO₂ injection - screw configuration 2 and extruder set up
2. Investigation of the effect of CO₂ pressure,
screw speed and feeding rate on the torque of the
extruder.

| | T₁-T₂ | T₃-T_{die}¹ | P₁ | P₂ | P₃ | N | F | T | Pₚᵤₘₚ |
|---|---|---|---|---|---|---|---|---|---|
| Nr. | (° C.) | (° C.) | (bar) | (bar) | (bar) | (rpm) | (kg/h) | (%) | (bar) |
| 1 | 180 | 120 | 35 | 31 | 34 | 100 | 1 | 78-98 | 35 |
| 2 | 180 | 120 | 36 | 33 | 35 | 100 | 1 | 73-92 | 40 |
| 3 | 180 | 120 | 33-42 | 30-38 | 33-41 | 100 | 1 | 73-93 | 45 |
| 4 | 180 | 120 | 23-45 | 19-42 | 22-44 | 100 | 1 | 73-96 | 50 |
| 5 | 180 | 120 | 28-50 | 25-46 | 24-48 | 100 | 1 | 71-100 | 60 |
| 6 | 180 | 120 | 35-41 | 33-40 | 35-41 | 100 | 1 | 85-100 | 70 |
| 7 | 180 | 120 | 36 | 33 | 36 | 100 | 1 | 74-93 | 38 |
| 8 | 180 | 120 | 36 | 33 | 36 | 150 | 1 | 69-87 | 38 |
| 9 | 180 | 120 | 30-35 | 27-32 | 30-34 | 200 | 1 | 64-85 | 38 |
| 10 | 180 | 120 | 36 | 33 | 36 | 100 | 0.5 | 66-86 | 39 |
| 11 | 180 | 120 | 31-36 | 28-33 | 31-35 | 100 | 1.0 | 76-95 | 39 |
| 12 | 180 | 120 | 35 | 32 | 40 | 100 | 1.5 | 85-100 | 39 |

¹T₃-T_{die}: all zones between T₃ and T_{die} were kept at the same temperature.

When doing experiments 1-6 it was observed that maximum foaming and steady state was obtained at 35-40 bar at temperature settings of 120° C., a screw speed of 100 rpm and a feeding rate of 1 kg/hr. A further increase of pressure resulted in less polymer foaming and periodic pressure drops. At 70 bar, pressure drops occurred continually. The torque was not influenced, except at pressures above 60 bar, where the torque increased.

Changing the screw speed (experiments 7-9 in Table 28) resulted in less polymer foaming and at 200 rpm, periodic pressure drops started to occur. Decreasing the feeding rate (experiments 10-12 in Table 28) also resulted in a lower extent of polymer foaming, while increasing the feeding rate did not influence polymer foaming or steady state. At 1.5 kg/hr, maximum torque was reached and thus this parameter could not be further explored. These observations were similar to what was observed during the trials with PVP-VA 64 and carbon dioxide injection.

A final set of experiments was performed to evaluate the effect of injecting CO₂ under CFR instead of CPR. Results are shown in Table 29. At 120° C., steady state was obtained below 4 ml/min. At a flow rate of 4 ml/min, pressure drops started to occur and less polymer foaming was observed. At 15 ml/min, pressure drops were occurring almost constantly accompanied by gas escape from the die. The torque was not influenced within the range of 0.5-15 ml/min.

TABLE 29
Parameter settings for extrusion experiments of Eudragit E100 PO with
CO₂ injection - screw configuration 2 and extruder set up 2. Investigation
of the effect of CFR on the torque of the machine at 120° C.

| | T₁-T₂ | T₃-T_{die1} | P1 | P2 | P3 | N | F | T | CFR |
|---|---|---|---|---|---|---|---|---|---|
| Nr. | (° C.) | (° C.) | (bar) | (bar) | (bar) | (rpm) | (kg/h) | (%) | (ml/min) |
| 1 | 180 | 120 | 38 | 34 | 36 | 100 | 1 | 74-90 | 0.5 |
| 2 | 180 | 120 | 37 | 34 | 36 | 100 | 1 | 75-94 | 1.0 |
| 3 | 180 | 120 | 38 | 35 | 37 | 100 | 1 | 75-93 | 2.0 |
| 4 | 180 | 120 | 35-39 | 31-36 | 33-39 | 100 | 1 | 74-93 | 4.0 |
| 5 | 180 | 120 | 34-43 | 30-40 | 33-42 | 100 | 1 | 77-95 | 8.0 |
| 6 | 180 | 120 | 28-47 | 25-44 | 27-46 | 100 | 1 | 76-95 | 10.0 |
| 7 | 180 | 120 | 34-44 | 31-40 | 33-42 | 100 | 1 | 74-95 | 12.0 |
| 9 | 180 | 120 | 37-49 | 34-46 | 36-48 | 100 | 1 | 76-95 | 15.0 |

¹T₃-T_{die}: all zones between T₃ and T_{die} were kept at the same temperature.

2.3. Physicochemical characteristics of Eudragit E100 before and after processing with CO₂

The physico-chemical properties of Eudragit E100 PO were examined before and after treatment with carbon dioxide, since processing with sub- or supercritical carbon dioxide could have induced changes to the polymer.

Modulated DSC was performed to investigate the thermal characteristics of Eudragit E100 PO. The glass transition was measured using the reversing DSC signal. The results are shown in Table 30. These results show that there is a difference in position of the glass transition before and after treatment when standard pans are used (t-test, n=3, P<0.05) . When the start and end point of the transition are considered (by respectively T₁ and T₂, being the temperatures at which the inflection of the baseline is observed), the range before treatment is smaller compared to the range after treatment, indicating that thermally responsive elements may have been altered with the polymer during the extrusion process. Depolymerisation may have occurred under the influence of heat or the structure of the polymeric chains may have been augmented. The position of the Tg was not different when measured in hermetically closed pan, but the ranges were somewhat different, confirming the results using standard pans.

TABLE 30
Glass transition of Eudragit E100 PO before and after treatment
with carbon dioxide. The samples were measured using modulated-DSC
and the glass transition is measured in the reversing signal.
Both standard and hermetically sealed pans were used. Experiments
were performed at least in duplicate.

| | Before treatment | | | After treatment | | |
|---|---|---|---|---|---|---|
| | (° C.) | | | (° C.) | | |
| | Tg | T₁ | T₂ | Tg | T₁ | T₂ |
| Standard pan | | | | | | |
| 1 | 51.68 | 50.39 | 52.60 | 47.50 | 40.81 | 54.99 |
| 2 | 52.33 | 49.28 | 53.52 | 49.03 | 39.89 | 56.47 |
| 3 | 51.32 | 46.89 | 52.60 | 49.89 | 43.76 | 56.65 |
| AVG | 51.78 | 48.85 | 52.91 | 48.81 | 41.49 | 56.04 |
| Hermetically | | | | | | |
| sealed pan | | | | | | |
| 1 | 48.65 | 47.81 | 50.02 | 47.40 | 42.47 | 50.94 |
| 2 | 48.80 | 47.60 | 53.89 | 49.31 | 47.81 | 51.31 |
| AVG | 48.73 | 47.71 | 51.96 | 48.36 | 45.14 | 51.13 |

TGA analysis was performed to measure any residual solvent loss. Table 31 shows that there is no difference in residual solvent before and after treatment.

TABLE 31
Residual solvent loss of Eudragit E100 PO before and after
treatment with carbon dioxide. The samples were measured using
TGA. Experiments were performed at least in quadruple.

| | Before treatment | After treatment |
|---|---|---|
| | Solvent loss (%) | Solvent loss (%) |
| 1 | 0.24 | 0.37 |
| 2 | 0.35 | 0.38 |
| 3 | 0.28 | 0.25 |
| 4 | 0.28 | 0.20 |
| 5 | 0.32 | — |
| AVG | 0.29 | 0.30 |

the dissolution of Eudragit E100 PO was measured to evaluate whether there was an effect on the dissolution profile after treatment with carbon dioxide. Comparison of the dissolution profiles when measured in 0.1 N HCl, showed that the sample treated with CO₂ dissolved faster at the 5, 15 and 30 minutes time point (t-test, n=3, P<0.05). After 45 minutes both samples were completely dissolved.

Interestingly, the same observations could be made for the dissolution experiments when performed in 0.01 N HCl (at the 5 and 15 minute time point based on a t-test, n=3, P<0.05) and thus this confirmed the results in 0.1 N HCl.

Particle size of the polymer before and after carbon dioxide treatment is shown in Table 32. Based on these results, the sample before treatment has a smaller average particle size compared to the carbon dioxide treated sample. According to the Noyes-Whitney equation (see Section I.), one would expect that a lower particle size would result in a faster dissolution and thus, the sample before treatment should dissolve more rapidly. However, according to FIGS. 19 and 20, this was not observed. As with PVP-VA 64, this faster dissolution rate of the treated samples may be attributed to a change in morphology through the foam formation when CO₂ is expanded at the exit of the extruder.

Therefore measurement of the specific surface area and microscopy (light microscopy and SEM) were performed on the samples.

TABLE 32
Particle size of Eudragit E100 PO before and after
treatment with carbon dioxide. The particle size
was measured using the vibrating sieve method.

| | Before treatment | After treatment |
|---|---|---|
| d84 | 67μ | 101μ |
| d50 | 138μ | 181μ |
| <100μ | 51.5% | 32.0% |

Measurement of the specific surface area showed that the unprocessed sample had a larger specific surface area (2.324 m²/g) compared to the processed sample (0.073 m²/g). This larger specific surface area for the unprocessed sample can be explained by the smaller particle size compared to the carbon dioxide-treated sample.

As with PVP-VA 64, the light photomicrographs showed that the foam consisted of thin walls connected to each other. Also the SEM figures clearly showed that the morphology of the carbon dioxide-treated polymer had changed from sphere like particles, before treatment, to platelets after treatment. These platelets were formed during the foaming step when CO₂ expanded at the exit of the extruder. These SEM pictures confirmed the differences in particle size and specific surface area before and after treatment of Eudragit E100.

2.4. Conclusion for Eudragit E100 PO

The extruder set up and screw configuration that were found to be optimal for PVP-VA 64, could also be used for Eudragit E100 PO. A method to obtain steady state conditions and significant polymer foaming upon release of the pressure could be identified for Eudragit E100 PO as well. This method consists of gradually decreasing the temperature in the barrel while increasing the pressure of the injected gas. Furthermore, it can be concluded that CO₂ acts as a plasticizer for Eudragit E100 since the process temperature can be lowered by at least 15° C. Steady state is obtained as a function of optimal pump pressure and temperature settings. The maximal pressure that could be obtained with Eudragit E100 PO was approximately 60 bar, which means that the extrusion in this case was also performed under subcritical conditions. The physicochemical characterization of the polymer revealed that the morphology was changed as a function of CO₂ treatments such that an increased dissolution of the polymer, both in 0.1 N HCl as well as in 0.01 N HCl, was observed. Also based on the measurement of the glass transition, a difference was observed after the extrusion and injection of carbon dioxide.

3. Milling Efficiency

In order to determine the efficiency of milling, extrudate examples that were extruded with and without carbon dioxide injection were resubjected to particle size determination.

25 g of each sample was milled for 30 seconds with a Bamix laboratory mill and particle size was obtained using the vibrating sieve method (amplitude 1.5 mm, 10 minutes).

Results:

| Sample | d84 (μ) | d50 (μ) | <100μ (%) |
|---|---|---|---|
| PVP-VA 64 before¹ | 236 | 542 | 5.6 |
| PVP-VA 64 after² | 74 | 154 | 44.9 |
| Eudragit E100 before | 438 | 764 | 1.2 |
| Eudragit E100 after | 172 | 415 | 8.9 |
| R51211/PVP-VA 64 10/90 before | 178 | 455 | 9.7 |
| R51211/PVP-VA 64 10/90 after | 123 | 176 | 27.6 |
| R51211/PVP-VA 64 40/60 before | 150 | 339 | 13.8 |
| R51211/PVP-VA 64 40/60 after | 237 | 578 | 4.2 |

¹melt extrudate sample before treatment with carbon dioxide
²melt extrudate sample after treatment with carbon dioxide

During sieve analysis for sample “R51211/PVP-VA 64 40/60 after”, it was observed that the 500 micron sieve was clogged with particles. This was probably due to electrostatic interaction between the particles, resulting in agglomeration during sieving. However, visual observation indicated that the particle size of this sample was smaller compared to the sample before carbon dioxide treatment. Therefore the samples were further analyzed by microscopic particle size analysis. This analysis showed that the particle size sample after treatment with carbon dioxide was smaller compared to before treatment. The median particle size for the extrudate treated with CO₂ was approximately 4 micron.

Conclusion:

These results show that the particle size of the extrudates treated with carbon dioxide is smaller compared to the extrudate samples prepared without carbon dioxide treatment. In other words, due to the injection of carbon dioxide during the melt extrusion process and the subsequent expansion and foaming, the extrudates are easier to mill.

### V. GENERAL CONCLUSIONS

Using PVP-VA 64 as a model polymer, an optimal extruder configuration and screw design were found which allowed for, (a) the injection of pressurized carbon dioxide, (b) the build up of pressure inside the extruder, (c) providing for an intimate contact between polymer and carbon dioxide so that the pressurized gas can dissolve in the polymer and (d) the formation of a foam upon expansion of the carbon dioxide. This configuration also provided a method to establish a condition of steady state (no pressure drops, i.e. no leakage of carbon dioxide) accompanied with significant polymer foaming upon release of the pressure. This method consisted of gradually decreasing the temperature in the barrel while increasing the pressure of the injected gas.

Furthermore, it can be concluded that CO₂ acts as a plasticizer for both PVP-VA 64 and Eudragit E100 PO since the processing temperature can be lowered by 30° C. and 15° C., respectively. Steady state is obtained as a function of optimal pump pressure and temperature settings. The maximal pressure that could be obtained with PVP-VA 64 was approximately 65 bar and, for Eudragit E100 PO 60 bar. This means that the extrusion was performed under subcritical conditions.

Foam formation was observed for both polymers, resulting in a significant change of the morphology, providing for increased dissolution of the polymer. For Eudragit E100, the position of the glass transition was changed.

### VI. REFERENCES

1. T. Gamse and Z. Knez, High Pressure Chemical Engineering Processes: Basics and Applications, Socrates Intensive Course, Graz, Austria, 2002.

2. M. A. McHugh and V. J. Krukonis, Supercritical Fluid Extraction: Principles and Practice, 2^{nd} Edition, Butterworth-Heinemann, Newton, Mass., USA, 1994.

3. R. Marentis, Supercritical Fluid Technology Seminar, ChemShow, N.Y., USA, 1999.

4. J. Jung and M. Perrut, Particle Design Using Supercritical Fluids: Literature and Patent Survey, J. Supercritical Fluids, 20 (3), 179-219, 2001.

5. R. A. Prentis, Y. Lis and S. R. Walker. Pharmaceutical innovation by seven UK-owned pharmaceutical companies (1964-1985). Br. J. Clin. Pharmacol. 25, 387-396, 1988.

6. C. A. Lipinski, F. Lombardo, B. W. Doriny and P. J. Feeney. Experimental and computational approaches to estimate solubility and permeability in drug discovery and development settings. Adv. Drug Deliv. Rev. 23, 3-25, 1997.

7. C. A. Lipinski. Avoiding investment in doomed drugs. Curr. Drug Disc. 1, 17-19, 2001.

8. P. Sheen, V. Khetarpal, C. Cariola, and C. Rowlings. Formulation of a poorly water-soluble drug in solid dispersions to improve bioavailability. Int. J. Pharm. 118, 221-227, 1995.

9. A. T. Serajuddin. Solid dispersion of poorly water-soluble drugs. Early promises, subsequent problems and recent breakthroughs. J. Pharm. Sci. 88, 1058-1066, 1999.

10. C. Leuner and J. Dressman. Improving drug solubility for oral delivery using solid dispersion. Eur. J. Pharm. Biopharm. 50,47-60, 2000.

11. H. H. Gruenhagen, Melt Extrusion Technology. Pharmaceut. Manufact. International 1995, 167-170.

12. J. Breitenbach, Melt extrusion: from process to drug deliver technology. Review article, Eur. J. Pharm. Biopharm., 54, 107-117, 2002.

13. C. Lefebvre, M. Brazier, H. Robert, A.M. Guyot-Hermann, Solid dispersions why and how? Industrial aspect., STP Pharma Sci., 4, 300-322, 1985.

14. T. Clifford, Fundamentals of Supercritical Fluids, Oxford University Press, NY, USA, 1999.

15. M. A. Winters, D. Z. Frankel, P. G. Debenedetti, J. Carey, M. Devaney, T. M. Przybycien, Protein purification with vapor-phase carbon dioxide. Biotec. Bioeng., 62, 247-258, 1999.

16. M. Perrut, Advances in Supercritical Fluid Chromatographic Processes, J. Chromatography, 658, 293-313, 1994.

17. J. H. Kim, T. E. Paxton, D. L. Tomasko, Microencapsulation of Naproxen Using Rapid Expansion of Supercritical Solutions, Biotechnol. Prog., 12, 650-661, 1996.

18. P. G. Debenedetti, J. W. Tom, S. D. Yeo, G. B. Lim, Application of supercritical fluids for the production of sustained delivery devices, J. Controlled Rel., 24, 27-44, 1993.

19. P. York, M. Hanna, B. Y. Shekunov, G. O. Humphreys, Microfine particle formation by SEDS: Scale up by design, Proceedings at Respiratory Drug Delivery, IL, USA, 1998.

20. J. Kerc, S. Srcic, Z. Knez, P. Sencar-Bozic, Micronisation of drugs using supercritical carbon dioxide, Int. J. Pharm., 182, 33-39, 1992.

21. R. W. Greiner, Pharmaceutically impregnated catheders, Eur. Pat., 0 405 284, 1991.

22. M. A. El-Egakey, M. Soliva, P. Speiser, Hot extruded dosage forms, Pharm. Acta Helv., 46, 31-52, 1971.

23. J. L. White, Twin Screw Extrusion, Technology and Principles. Hanser Publishers, New York, 1990.

24. L. P. B. M. Janssen, Verwerkingstechnologie van Polymeren, van halffabrikaat tot product, PAON, Den Dolder, the Netherlands, 1997.

25. L. Baert, D. Thone, and G. Verreck. Antifungal compositions with improved bioavailability, WO 9744014, 1997.

26. G. Verreck, L. Baert, J. Peeters and M. Brewster. Improving aqueous solubility and bioavailability for itraconazole by solid dispersion approach. AAPS PharmSci 3 (No. 3): M2157, 2001.

27. G. Verreck, K. Six, G. Van den Mooter, L. Baert, J. Peeters, M. E. Brewster, Characterization of Solid Dispersions of Itraconazole and Hydroxypropylmethylcellulose prepared by melt extrusion, Part 1, Int. J. Pharm., 251 (2003) 165-174.

28. Six K., Berghmans H., Leuner C., Dressman J., Van Werde K., Mullens J., Benoist L., Thimon M., Meublat L., Verreck G., Peeters J., Brewster M., Van den Mooter G., Characterization of solid dispersions of itraconazole and hydroxypropylmethylcellulose prepared by melt extrusion—Part II; Pharm. Res., 20, 7, (2003)1047-1054.

29. J. S. Chiou, J. W. Barlow, D. R. Paul, Plasticization of glassy polymers by CO₂, J. Appl. Polym. Sci., 30, 2633-2642, 1985.

30. M. D. Elkovitch, L. J. Lee, D. L. Tomasko, Viscosity reduction of polymers by the addition of supercritical carbon dioxide in polymer processing, ANTEC, 1407-1410, 1998.

31. M. D. Elkovitch, D. L. Tomasko, L. J. Lee, Supercritical carbon dioxide assisted blending of polystyrene and poly(methyl methacrylate), Polym. Eng. Sci., 39 (10), 2075, 1999.

32. M. Lee, C. Tzoganakis, C. B. Park, Extrusion of PE/PS Blends With Supercritical Carbon Dioxide, Polym. Eng. Sci., 38, 1112-1120, 1998.

33. C. B. Park, D. F. Baldwin, N. P. Suh, Effect of Pressure Drop Rate on Cell Nucleation in Continuous Processing of Microcellular Polymers, Polym. Eng. Sci., 35, 432-440, 1995.

34. Leistritz, Operating Instructions, Micro 18, Nurnberg, Germany, 2002.

35. TA Instruments Differential Scanning Calorimetry Training Course, TA Instruments, Brussels, Belgium, 2002.

36. Brunauer S., Emett P. H. and Teller E., Adsorption of gases in multimolecular layers, J. Am. Chem. Soc., 60, 309, 1938.

37. Volker Buhler, Polyvinylpyrrolidone for the pharmaceutical industry, BASF Aktiengesellschaft, August 1992.

38. Personal communication Prof. D. Tomasko, Ohio State University, Columbus, Ohio, 2003.

1. Particles of the polymer PVP-VA-60 or the polymer Eudragit-E100-PO, characterized in that said particles are shaped as platelets.

2. Particles of the polymer PVP-VA-60 according to claim 1 wherein the specific surface area is larger than 0.350 m²/g.

3. Particles of the polymer Eudragit-E100-PO according to claim 1 wherein less than 40% (w/w) is smaller than 100 μ.

4. Particles comprising the polymer PVP-VA-60 or the polymer Eudragit-E100-PO, and an active ingredient, characterized in that said particles are shaped as platelets.

5. Particles according to claim 4 wherein the active ingredient is itraconazole.

6. Particles according to claim 5 wherein the weight by weight ratio of itraconazole to polymer ranges from about 10/90 to about 40/60.

7. A pharmaceutical dosage form comprising a therapeutically effective amount of particles as defined in claims 4 to 6.

8. A process of preparing a pharmaceutical dosage form as defined in claim 7 comprising the steps of intimately mixing particles as defined in claims 4 to 6 with pharmaceutically acceptable excipients and making from the thus obtained mixture a pharmaceutical dosage form comprising a therapeutically effective amount of particles.

9. A process of preparing particles as defined in claim 1 or claim 4 comprising the steps of
feeding the polymer, or a mixture of the polymer and the active ingredient, into a melt extruder,
transporting the polymer, or a mixture of the polymer and the active ingredient, through the barrel of the melt extruder by means of a screw modified with transport elements and with kneading elements,
injecting pressurized gas into the barrel of the melt extruder through a port located in the barrel,
mixing the polymer, or a mixture of the polymer and the active ingredient, and the pressurized gas under subcritical or supercritical conditions
expanding the polymer, or a mixture of the polymer and the active ingredient, after the die plate, and
milling the extrudate,
characterized by creating a melt seal before the site of the pressurized gas injection by placing a reversing transport element in the screw configuration at said site.
